(19) United States
(12) Patent Application Publication
(10) Pub. No.: US 2023/0313094 A1
(43) Pub. Date: Oct. 5, 2023

(54) BIOPROCESSING SYSTEMS AND METHODS FOR SCALABLE MASS PRODUCTION OF MINICELLS

(71) Applicant: AgroSpheres, Inc., Charlottesville, VA (US)

(72) Inventors: Ameer Hamza SHAKEEL, Charlottesville, VA (US); Joseph FRANK, Charlottesville, VA (US); Jacob ENGLAENDER, Charlottesville, VA (US); Sepehr ZOMORODI, Charlottesville, VA (US); Payam POURTAHERI, Charlottesville, VA (US)

(21) Appl. No.: 18/332,559

(22) Filed: Jun. 9, 2023

Related U.S. Application Data
(63) Continuation of application No. PCT/US2021/062964, filed on Dec. 10, 2021.
(60) Provisional application No. 63/124,559, filed on Dec. 11, 2020.

(51) Int. Cl. C12M 3/00; C12M 1/34

(52) U.S. Cl. CPC C12M 21/08; C12M 41/36; C12M 41/42

(57) ABSTRACT

The present disclosure provides controlled bioprocessing systems for enhanced minicell production with defined fermentation parameters and methods of said bioprocessing systems and methods for the production of minicells from said bioprocessing systems using a bioreactor or fermenter.

### CROSS-REFERENCE TO RELATED APPLICATIONS

This application claims the benefit of priority to U.S. Provisional Application No. 63/124,559 filed on Dec. 11, 2020, which is hereby incorporated by reference in its entirety.

### FIELD OF THE DISCLOSURE

The present disclosure generally relates to bioprocessing systems and methods and, more particularly, to a bioprocessing system and methods for the production of minicells from a bioreactor system.

### BACKGROUND OF THE DISCLOSURE

The high quality and large quantity of biomaterials produced by a bioprocess system is critical in order to achieve a reliable, robust and economic manufacturing procedure when producing biomaterials from a bioreactor.

Increasing quality and maintaining product comparability while maximizing productivity in mass production creates challenges in bioprocess development. Media composition affects cell growth, productivity, and posttranslational modifications in cells of interest for mass production.

However, there are not many investigation on the effects of culture media on cell performance and product quality in terms of minicell productions despite the minicells are being developed as an important delivery system for human therapeutics and diagnostics, vaccines, chemical agents, agricultural compounds, and biologically active agents.

Thus, there is a need to introduce a bioprocess for optimizing the bioprocess to achieve the desired outcome of the minicell product in real time and in mass production. The results can be safer, more energy-efficient, and environmentally sustainable manufacturing bioprocesses for minicell production.

### SUMMARY OF THE DISCLOSURE

The present disclosure provides a bioprocessing system for minicell production, comprising: (a) at least one bioreactor; (b) a minimal medium for minicell production; and (c) at least one bacterial cell strain capable of producing a population of achromosomal minicells. In embodiments, said bioreactor is arranged for maintaining a continuous bioprocess configured to provide said population of achromosomal minicells In embodiments, said bioreactor is set with a fermentation parameter selected from the group of a feed rate, temperature, ingredients, dissolved oxygen, agitation speed, airflow rate, oxygen, pH, inoculum, and fermentation length. In embodiments, a minicell production yield in said bioprocessing system is at least 1.1 fold higher than a minicell production yield in an uncontrolled bioprocessing system. In embodiments, said feed rate is 0 to about 10 mL/min/L. In embodiments, said temperature is from about 10° C. to about 70° C. In embodiments, said ingredients comprises a carbon source, a trace metal, a vitamin, a buffer, a nitrogen source, an antifoam, an additional growth promoting ingredient. In embodiments, said dissolved oxygen is 0 to 100%. In embodiments, said agitation speed is about 50 to about 10,000 rpm. In embodiments, said air flow rate is about 0.1 to about 20 standard liters per minute (SLPM). In embodiments, said oxygen is 0 to 100%. In embodiments, said pH is about 3 to about 10. In embodiments, said inoculum is about 0.1 to about 20%. In embodiments, said fermentation length is about 12 to about 200 hours. In embodiments, said minicell is about 150 nm to about 950 nm in length. In embodiments, said carbon source is a glycerol or a glucose. In embodiments, said uncontrolled bioprocessing system is an incubator system or a shaker flask system. In embodiments, a minicell ratio after said bioprocessing is at least 40% of total cells in a bioreactor.

The present disclosure teaches that minicells produced from the bioprocessing system taught herein is capable of encapsulating an agricultural agent. In embodiments, said agricultural agent is an agrochemical compound or a biologically active compound. In embodiments, said agrochemical compound is selected from the group consisting of: a pesticide, an herbicide, an insecticide, a fungicide, a nematicide, a fertilizer and a hormone or a chemical growth agent. In embodiments, said biologically active compound is selected from a nucleic acid, a peptide, a protein, an essential oil, and combinations thereof. In embodiments, the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof. In embodiments, the essential oil comprises geraniol, eugenol, genistein, carvacrol, thymol, pyrethrum or carvacrol.

The present disclosure provides that the bioprocessing system is a controlled continuous bioprocessing system capable of continuously producing a population of achromosomal minicells. In embodiments, said produced minicells are partially harvested and said bioprocessing system continuously run to produce another population of achromosomal minicells. In embodiments, said minicells is partially harvested from about 5% to about 90% of total cells in said bioreactor.

The present disclosure provides a method of bioprocessing, comprising the steps of: (a) introducing at least one bacterial cell strain into a bioreactor setting comprising minimal media; (b) culturing said bacterial cell strain from (a) to produce a population of achromosomal minicells in said bioreactor setting, wherein said bacterial cell strain is a minicell-producing bacterial cell strain, wherein said population of achromosomal minicells are produced from step (b), and wherein said bioreactor setting is configured with a fermentation parameter selected from the group of a feed rate, temperature, ingredients, dissolved oxygen, agitation speed, airflow rate, oxygen, pH, inoculum, and fermentation length, as listed in Table 1. In embodiments, the method further comprises the steps of: (c) harvesting a batch of cells comprising said bacterial cells and a population of newly-produced minicells from step (b); (d) purifying said batch of cells; (e) filtering or sorting out said population of achromosomal minicells from said batch of cells; and (f) concentrating said minicells. In embodiments, the purifying is performed by disc stack centrifugation. In embodiments, said concentrated minicells are stored as a liquid form or a powder form. In embodiments, said powder form is prepared by freeze-drying, vacuum drying, or heat drying of said concentrated minicells. In embodiments, said bioprocessing is a controlled continuous bioprocessing capable of continuously producing a population of achromosomal minicells. In embodiments, said produced minicells are partially harvested and said bioprocessing system continuously run to produce another population of achromosomal minicells. In embodiments, said minicells is partially harvested from about 5% to about 90% of total cells in said bioreactor.

The present disclosure provide minicells produced by the methods described herein. In some embodiments, said minicell is produced in said bioprocessing setting at least 1.1 fold higher than a minicell production in an uncontrolled bioprocessing setting.

### BRIEF DESCRIPTION OF THE FIGURES

FIGS. 1A-1D show time course of minicell production from P6* bacterial strain at hours 1, 2, 3, 16 in an uncontrolled bioprocess system using a shaker. The strain was grown in a shaker flask with Lysogeny Broth (LB), Terrific Broth (TB) or similar complex media at 37° C. First peak at ˜0.5 um capturing minicells and second peak at >1 um capturing parent bacterial cells. Each graph (FIGS. 1A-1D) represents data from n=4 replicated runs. Minicell production was measured at hour 1 (FIG. 1A), hour 2 (FIG. 1B), hour 3 (FIG. 1C), and hour 16 (FIG. 1D).

FIG. 1E demonstrates actual numbers of experimental data displayed from FIGS. 1A-1D, including minicell size range (nm), total minicells, average minicell length (nm), parental bacterial size range (nm), total bacterial cells, average bacteria length (nm), total objects, and minicell ratio (%) at different incubation times (1 hour; 2 hours; 3 hours; and 16 hours).

FIGS. 2A-2D show four individual runs for minicell production from P6* bacterial strain in a controlled bioprocess system using a bioreactor/fermenter. The strain was grown in a bioreactor with defined minimal media at 37° C. First peak at ˜0.5 um capturing minicells and second peak at >1 um capturing parent bacterial cells. Each graph (FIGS. 2A-2D) represents data from n=4 replicated runs. Each data was collected after completion of four individual fermentation runs at 36 hours of fermentation. Minicell production was measured from four independent controlled bioprocess runs on different days; Run 1 (FIG. 2A), Run 2 (FIG. 2B), Run 3 (FIG. 2C), and Run 4 (FIG. 2D).

FIG. 2E demonstrates actual numbers of experimental data displayed from FIGS. 2A-2D, including minicell size range (nm), total minicells, average minicell length (nm), parental bacterial size range (nm), total bacterial cells, average bacteria length (nm), total objects, and minicell ratio (%) from four individual runs of 36 hour-fermentation.

FIG. 3 shows a table presenting enhanced minicell production retained in a variety of parameter combinations of the controlled bioprocessing system using the bioreactor. The parameters tested for combination are Oxygen Transfer (as measured by Oxygen Uptake Rate: OUR), airflow (VVM), and presence of oxygen (02).

FIGS. 4A-4C show effect of various parameters on minicell production. Oxygen transfer (as measured by OUR) vs minicell production yield (FIG. 4A), airflow (VVM) vs minicell production yield (FIG. 4B) and supply of oxygen (02) vs minicell production yield (FIG. 4C).

FIG. 5 shows effect of temperature on minicell production from P826 strain.

FIGS. 6A-6B show comparison of minicell production from P826 strain between two bioprocessing systems (uncontrolled vs controlled). The uncontrolled bioprocess system (FIG. 6A) was performed in a shaker that was unable to constantly control given parameters. The controlled bioprocess system was automatically controlled to maintain given parameters (pH at 6.7, dissolved oxygen (DO) at about 30%, and temperature at 25° C.).

FIGS. 6C-6D show comparison of minicell production from P826 strain using the same setting of FIGS. 6A-6B except that the temperature was set at 35° C.

FIGS. 7A-7D show minicell production from minicell-producing bacteria strains that also express biomolecule (RNA/Protein/metabolite) in a controlled bioprocessing system within the defined parameter ranges. Different bacterial cell lines were tested for minicell production; minicell-producing P8-T7 cell strain (FIG. 7A); R1 strain derived from BL21-AI carrying a construct expressing a fluorescent dsRNA hairpin (FIG. 7B); minicell-producing R1 strain carrying a construct expressing dsRNA to control insects (FIG. 7C); R1 strain carrying a construct expressing dsRNA to control fungi (FIG. 7D).

FIG. 7E shows minicell production from a minicell-producing bacteria strain (R1) carrying a construct expressing dsRNA to control insects in a controlled bioprocessing system, but outside of the defined parameter ranges of the present disclosure.

### DETAILED DESCRIPTION

To maintain high quality and quantity of minicell production with maximizing productivity, a new bioprocessing system are required to ensure a safe, scalable, energy-efficient, environmentally sustainable, and cost-effective manufacturing bioprocesses for minicell production.

The present disclosure relates to a controlled production of minicells using a bioreactor/fermenter system. The present disclosure also relates to a method for optimizing the bioprocess to achieve the desired outcome of the minicell product in mass production.

### Definitions

While the following terms are believed to be well understood by one of ordinary skill in the art, the following definitions are set forth to facilitate explanation of the presently disclosed subject matter.

The term “a” or “an” refers to one or more of that entity, i.e. can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein the terms “cellular organism” “microorganism” or “microbe” should be taken broadly. These terms are used interchangeably and include, but are not limited to, the two prokaryotic domains, Bacteria and Archaea, as well as certain eukaryotic fungi and protists.

The term “prokaryotes” is art recognized and refers to cells that contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

The terms “genetically modified host cell,” “recombinant host cell,” and “recombinant strain” are used interchangeably herein and refer to host cells that have been genetically modified by the cloning and transformation methods of the present disclosure. Thus, the terms include a host cell (e.g., bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring organism from which it was derived. It is understood that in some embodiments, the terms refer not only to the particular recombinant host cell in question, but also to the progeny or potential progeny of such a host cell.

The term “wild-type microorganism” or “wild-type host cell” describes a cell that occurs in nature, i.e. a cell that has not been genetically modified. In the disclosure, “wild type strain” or “wild strain” or “wild type cell line” refers to a cell strain/line that can produce minicells. In some embodiments, wild type bacterial strains and/or cell lines such as E. coli strain p678-54 and B. subtilis strain CU403 can make miniature cells deficient in DNA. Methods for producing such minicells are known in the art. See, for example, Adler et al., 1967, Proc. Natl. Acad. Sci. USA 57:321-326; Inselburg J, 1970 J. Bacteriol. 102(3):642-647; Frazer 1975, Curr. Topics Microbiol. Immunol. 69:1-84, Reeve et al 1973, J. Bacteriol. 114(2):860-873; and Mendelson et al 1974 J. Bacteriol. 117(3): 1312-1319.

The term “genetically engineered” may refer to any manipulation of a host cell's genome (e.g. by insertion, deletion, mutation, or replacement of nucleic acids).

As used herein, the term “genetic engineering” refers to techniques for the genetic manipulation of one or more cells, whereby the genome of the one or more cells has been modified, inserted, deleted, mutated, or replaced by at least one DNA sequence. Candidate DNA sequences include but are not limited to genes that are not naturally present, DNA sequences that are not normally transcribed into RNA or translated into a protein (“expressed”), and other genes or DNA sequences which one desires to introduce into the one or more cells.

As used herein, “gene editing” or “genome editing” is a technique in which endogenous chromosomal sequences present in one or more cells within a subject, can be edited, e.g., modified, using targeted endonucleases and single-stranded nucleic acids. The genome editing method can result in the insertion of a nucleic acid sequence at a specific region within the genome, the excision of a specific sequence from the genome and/or the replacement of a specific genomic sequence with a new nucleic acid sequence. In certain embodiments, the genome editing technique can results in the repression of the expression of a gene. For example, and not by way of limitation, a nucleic acid sequence can be inserted at a chromosomal breakpoint of a fusion gene. A non-limiting example of a genome editing technique for use in the disclosed methods is the CRISPR system, e.g., CRISPR/Cas 9 system. Non-limiting examples of such genome editing techniques are disclosed in PCT Application Nos. WO 2014/093701 and WO 2014/165825, the contents of which are hereby incorporated by reference in their entireties. In some embodiments, the genome editing technique can include the use of one or more guide RNAs (gRNAs), complementary to a specific sequence within a genome (i.e. a target), including protospacer adjacent motifs (PAMs), to guide a nuclease, e.g., an endonuclease, to the specific genomic sequence. In certain embodiments, the genome editing technique can include the use of one or more guide RNAs (gRNAs), complementary to the sequences that are adjacent to and/or overlap the target, to guide one or more nucleases.

The term “control” or “control host cell” refers to an appropriate comparator host cell for determining the effect of a genetic modification or experimental treatment. In some embodiments, the control host cell is a wild type cell. In other embodiments, a control host cell is genetically identical to the genetically modified host cell, save for the genetic modification(s) differentiating the treatment host cell.

As used herein, the term “allele(s)” means any of one or more alternative forms of a gene, all of which alleles relate to at least one trait or characteristic. In a diploid cell, the two alleles of a given gene occupy corresponding loci on a pair of homologous chromosomes.

As used herein, the term “locus” (loci plural) means a specific place or places or a site on a chromosome where for example a gene or genetic marker is found.

As used herein, the term “genetically linked” refers to two or more traits that are co-inherited at a high rate during breeding such that they are difficult to separate through crossing.

A “recombination” or “recombination event” as used herein refers to a chromosomal crossing over or independent assortment.

As used herein, the term “phenotype” refers to the observable characteristics of an individual cell, cell culture, organism, or group of organisms which results from the interaction between that individual's genetic makeup (i.e., genotype) and the environment.

As used herein, the term “chimeric” or “recombinant” when describing a nucleic acid sequence or a protein sequence refers to a nucleic acid, or a protein sequence, that links at least two heterologous polynucleotides, or two heterologous polypeptides, into a single macromolecule, or that rearranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

As used herein, a “synthetic amino acid sequence” or “synthetic peptide” or “synthetic protein” is an amino acid sequence that is not known to occur in nature or that is not naturally occurring. Generally, such a synthetic protein sequence will comprise at least one amino acid difference when compared to any other naturally occurring protein sequence.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. An example of a local alignment algorithm utilized for the comparison of sequences is the NCBI Basic Local Alignment Search Tool (BLAST®) (Altschul et al. 1990 J. Mol. Biol. 215: 403-10), which is available from several sources, including the National Center for Biotechnology Information (NCBI, Bethesda, Md.) and on the Internet, for use in connection with the sequence analysis programs blastp, blastn, blastx, tblastn and tblastx. It can be accessed on the internet via the National Library of Medicine (NLM)'s world-wide-web URL. A description of how to determine sequence identity using this program is available at the NLM's website on BLAST tutorial. Another example of a mathematical algorithm utilized for the global comparison of sequences is the Clustal W and Clustal X (Larkin et al. 2007 Bioinformatics, 23, 2947-294, Clustal W and Clustal X version 2.0) as well as Clustal omega. Unless otherwise stated, references to sequence identity used herein refer to the NCBI Basic Local Alignment Search Tool (BLAST®).

As used herein, the term “endogenous” or “endogenous gene,” refers to the naturally occurring gene, in the location in which it is naturally found within the host cell genome. In the context of the present disclosure, operably linking a heterologous promoter to an endogenous gene means genetically inserting a heterologous promoter sequence in front of an existing gene, in the location where that gene is naturally present. An endogenous gene as described herein can include alleles of naturally occurring genes that have been mutated according to any of the methods of the present disclosure.

As used herein, the term “exogenous” is used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode an enzymatically active portion of a genetic regulatory element. An enzymatically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458.

For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3rd ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

As used herein, “promoter” refers to a DNA sequence capable of controlling the expression of a coding sequence or functional RNA. In some embodiments, the promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, the phrases “recombinant construct”, “expression construct”, “chimeric construct”, “construct”, and “recombinant DNA construct” are used interchangeably herein. Also, “construct”, “vector”, and “plasmid” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

“Operably linked” means in this context the sequential arrangement of the promoter polynucleotide according to the disclosure with a further oligo- or polynucleotide, resulting in transcription of said further polynucleotide.

As used herein, the term “display” refers to the exposure of the polypeptide of interest on the outer surface of the minicell. By way of non-limiting example, the displayed polypeptide may be a protein or a protein domain which is either expressed on the minicell membrane or is associated with the minicell membrane such that the extracellular domain or domain of interest is exposed on the outer surface of the minicell (expressed and displayed on the surface of the minicell or expressed in the parental cell to be displayed on the surface of the segregated/budded minicell). In all instances, the “displayed” protein or protein domain is available for interaction with extracellular components. A membrane-associated protein may have more than one extracellular domain, and a minicell of the disclosure may display more than one membrane-associated protein.

As used herein, the terms “polypeptide”, “protein” and “protein domain” refer to a macromolecule made up of a single chain of amino acids joined by peptide bonds. Polypeptides of the disclosure may comprise naturally occurring amino acids, synthetic amino acids, genetically encoded amino acids, non-genetically encoded amino acids, and combinations thereof. Polypeptides may include both L-form and D-form amino acids.

As used herein, the term “enzymatically active polypeptide” refers to a polypeptide which encodes an enzymatically functional protein. The term “enzymatically active polypeptide” includes but not limited to fusion proteins which perform a biological function. Exemplary enzymatically active polypeptides, include but not limited to enzymes/enzyme moiety (e.g. wild type, variants, or engineered variants) that specifically bind to certain receptors or biological/chemical substrates to effect a biological function such as biological signal transduction or chemical inactivation.

As used herein, the term “protease-deficient strain” refers to a strain that is deficient in one or more endogenous proteases. For example, protease deficiency can be created by deleting, removing, knock-out, silencing, suppressing, or otherwise downregulating at lease on endogenous protease. Said proteases can include catastrophic proteases. For example, BL21 (DE3) E. coli strain is deficient in proteases Lon and OmpT. E. coli strain has cytoplasmic proteases and membrane proteases that can significantly decrease protein production and localization to the membrane. In some embodiments, a protease-deficient strain can maximize production and localization of a protein of interest to the membrane of the cell. “Protease-deficient” can be interchangeably used as “protease-free” in the present disclosure.

As used herein, the term “anucleated cell” refers to a cell that lacks a nucleus and also lacks chromosomal DNA and which can also be termed as an “anucleate cell”. Because eubacterial and archaebacterial cells, unlike eukaryotic cells, naturally do not have a nucleus (a distinct organelle that contains chromosomes), these non-eukaryotic cells are of course more accurately described as being “without chromosomes” or “achromosomal.” Nonetheless, those skilled in the art often use the term “anucleated” when referring to bacterial minicells in addition to other eukaryotic minicells. Accordingly, in the present disclosure, the term “minicells” encompasses derivatives of eubacterial cells that lack a chromosome; derivatives of archaebacterial cells that lack their chromosome(s), and anucleate derivatives of eukaryotic cells that lack a nucleus and consequently a chromosome. Thus, in the present disclosure, “anucleated cell” or “anucleate cell” can be interchangeably used with the term “achromosomal cell.”

As used herein, the term “non-expressed” agricultural compound refers to an agricultural compound that is not endogenously, innately or naturally produced from a host cell. For example, Bacillus thurigenesis produces a toxin that can kill plant chewing insect larvae as well as mosquito larvae. This toxin that can be used as an agricultural compound is a Cry protein endogenously expressed from B. thurigenesis. In the present disclosure, this naturally expressed Cry protein is not considered as a non-expressed agricultural compound.

As used herein, “carrier,” “acceptable carrier,” or “agriculturally acceptable carrier” refers to a diluent, adjuvant, excipient, or vehicle with which a composition can be administered to its target, which does not detrimentally effect the composition.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid,” “nucleotide,” and “polynucleotide” are used interchangeably.

As used herein, the term “protease-deficient strain” refers to a strain that is deficient in one or more endogenous proteases. For example, protease deficiency can be created by deleting, removing, knock-out, silencing, suppressing, or otherwise downregulating at lease on endogenous protease. Said proteases can include catastrophic proteases. For example, BL21 (DE3) E. coli strain is deficient in proteases Lon and OmpT. E. coli strain has cytoplasmic proteases and membrane proteases that can significantly decrease protein production and localization to the membrane. In some embodiments, a protease-deficient strain can maximize production and localization of a protein of interest to the membrane of the cell. “Protease-deficient” can be interchangeably used as “protease-free” in the present disclosure.

As used herein, the term “ribonuclease-deficient strain” refers to a strain that is deficient in one or more endogenous ribonuclease. For example, ribonuclease deficiency can be created by deleting, removing, knock-out, silencing, suppressing, or otherwise downregulating at lease on endogenous ribonuclease. Said ribonuclease can include ribonuclease III. For example, HT115 E. coli strain is deficient in RNase III. In some embodiments, a ribonuclease-deficient strain is unable to and/or has a reduced capability of recognizing dsRNA and cleaving it at specific targeted locations. “Ribonuclease-deficient” can be interchangeably used as “ribonuclease-free” in the present disclosure.

As used herein, the term “achromosomal cell “anucleated cell” refers to a cell that lacks a nucleus and also lacks chromosomal DNA and which can also be termed as an “anucleate cell”. Because eubacterial and archaebacterial cells, unlike eukaryotic cells, naturally do not have a nucleus (a distinct organelle that contains chromosomes), these non-eukaryotic cells are of course more accurately described as being “without chromosomes” or “achromosomal.” Nonetheless, those skilled in the art often use the term “anucleated” when referring to bacterial minicells in addition to other eukaryotic minicells. Accordingly, in the present disclosure, the term “minicells” encompasses derivatives of eubacterial cells that lack a chromosome; derivatives of archaebacterial cells that lack their chromosome(s), and anucleate derivatives of eukaryotic cells that lack a nucleus and consequently a chromosome. Thus, in the present disclosure, “anucleated cell” or “anucleate cell” can be interchangeably used with the term “achromosomal cell.”

As used herein, the term “binding site,” means a molecular structure or compound, such as a protein, a polypeptide, a polysaccharide, a glycoprotein, a lipoprotein, a fatty acid, a lipid or a nucleic acid or a particular region in such molecular structure or compound or a particular conformation of such molecular structure or compound, or a combination or complex of such molecular structures or compounds. In certain embodiments, at least one binding site is on an intact living plant. An “intact living plant,” as used herein, means a plant as it grows, whether it grows in soil, in water or in artificial substrate, and whether it grows in the field, in a greenhouse, in a yard, in a garden, in a pot or in hydroponic culture systems. An intact living plant preferably comprises all plant parts (roots, stem, branches, leaves, needles, thorns, flowers, seeds etc.) that are normally present on such plant in nature, although some plant parts, such as, e.g., flowers, may be absent during certain periods in the plant's life cycle.

A “binding domain,” as used herein, means the whole or part of a proteinaceous (protein, protein-like or protein containing) molecule that is capable of binding using specific intermolecular interactions to a target molecule. A binding domain can be a naturally occurring molecule, it can be derived from a naturally occurring molecule, or it can be entirely artificially designed. A binding domain can be based on domains present in proteins, including but not limited to microbial proteins, protease inhibitors, toxins, fibronectin, lipocalins, single-chain antiparallel coiled coil proteins or repeat motif proteins. Non-limiting examples of such binding domains are carbohydrate binding modules (CBM) such as cellulose binding domain to be targeted to plants. In some embodiments, a cell adhesion moiety comprises a binding domain.

As used herein, “carrier,” “acceptable carrier,” or “biologically actively acceptable carrier” refers to a diluent, adjuvant, excipient, or vehicle with which a composition can be administered to its target, which does not detrimentally effect the composition.

As used herein, “plants” and “plant derivatives” can refer to any portion of a growing plant, including the roots, stems, stalks, leaves, branches, seeds, flowers, fruits, and the like. For example, cinnamon essential oil can be derived from the leaves or bark of a cinnamon plant.

As used herein, the term “essential oils” refers to aromatic, volatile liquids extracted from plant material. Essential oils are often concentrated hydrophobic liquids containing volatile aroma compounds. Essential oil chemical constituents can fall within general classes, such as terpenes (e.g., p-Cymene, limonene, sabinene, a-pinene, y-terpinene, b-caryophyllene), terpenoids (e.g., geraniol, citronellal, thymol, carvacrol, carvone, borneol) and phenylpropanoids (e.g., cinnamaldehyde, eugenol, vanillin, safrole). Essential oils can be natural (i.e., derived from plants), or synthetic.

As used herein, the term “essential oil” encompasses within the scope of the present disclosure also botanical oils and lipids. Non-limiting examples of essential oils are sesame oil, pyrethrum (extract), glycerol-derived lipids or glycerol fatty acid derivatives, cinnamon oil, cedar oil, clove oil, geranium oil, lemongrass oil, angelica oil, mint oil, turmeric oil, wintergreen oil, rosemary oil, anise oil, cardamom oil, caraway oil, chamomile oil, coriander oil, guaiacwood oil, cumin oil, dill oil, mint oil, parsley oil, basil oil, camphor oil, cananga oil, citronella oil, eucalyptus oil, fennel oil, ginger oil, copaiba balsam oil, perilla oil, cedarwood oil, jasmine oil, palmarosa sofia oil, western mint oil, star anis oil, tuberose oil, neroli oil, tolu balsam oil, patchouli oil, palmarosa oil, Chamaecyparis obtusa oil, Hiba oil, sandalwood oil, petitgrain oil, bay oil, vetivert oil, bergamot oil, Peru balsam oil, bois de rose oil, grapefruit oil, lemon oil, mandarin oil, orange oil, oregano oil, lavender oil, Lindera oil, pine needle oil, pepper oil, rose oil, iris oil, sweet orange oil, tangerine oil, tea tree oil, tea seed oil, thyme oil, thymol oil, garlic oil, peppermint oil, onion oil, linaloe oil, Japanese mint oil, spearmint oil, giant knotweed extract, and others as disclosed herein throughout.

As used herein, the term “complex medium” or “complex media” refers to a nutritionally rich medium primarily used for cell culturing (e.g. growth of bacteria), whose ingredients comprises tryptone and yeast extract. Complex media contain complex and expensive sources of carbon and nitrogen such as tryptone and yeast extract. Tryptone is a source of essential amino acids such as peptides and peptones to the growing bacteria and the yeast extract is used to provide a plethora of organic compounds helpful for bacterial growth. Lysogeny Broth (LB) and Terrific Broth (TB) media are examples of complex media, which are the most popular and commonly used media to culture bacteria.

As used herein, the term “minimal medium” or “defined minimal medium” refers to a medium used for cell culturing, but it contains commercially viable sources of carbon and nitrogen (e.g. glucose or glycerol as carbon source and ammonium phosphate as nitrogen source), which are more cost-feasible than yeast extract and tryptone used for the complex medium described above. A minimal media or media composed of the cost-feasible carbon and nitrogen sources is essential for biomaterial (e.g. minicell) manufacturing in an economical manner. In embodiments, the minimal medium is utilized for achromosomal (or anucleated) minicell production in a controlled bioprocessing system of the present disclosure using a small-, medium-, or large-scale of bioreactors with defined parameter ranges described herein.

Bacterial Minicell Production

Minicells are produced by parent cells having a mutation in, and/or overexpressing, or under expressing a gene involved in cell division and/or chromosomal partitioning, or from parent cells that have been exposed to certain conditions, that result in aberrant fission of bacterial cells and/or partitioning in abnormal chromosomal segregation during cellular fission (division). The term “parent cells” or “parental cells” refers to the cells from which minicells are produced. Minicells, most of which lack chromosomal DNA (Mulder et al., Mol Gen Genet, 221: 87-93, 1990), are generally, but need not be, smaller than their parent cells. Typically, minicells produced from E. coli cells are generally spherical in shape and are about 0.1 to about 0.5 μm in diameter and less than 1 μm in length, whereas whole E. coli cells are about from about 1 to about 3 μm in diameter and from about 2 to about 10 μm in length. Micrographs of E. coli cells and minicells that have been stained with DAPI (4:6-diamidino-z-phenylindole), a compound that binds to DNA, show that the minicells do not stain while the parent E. coli are brightly stained. Such micrographs demonstrate the lack of chromosomal DNA in minicells. (Mulder et al., Mol. Gen. Genet. 221:87-93, 1990).

Minicells are achromosomal, membrane-encapsulated biological nanoparticles (1 μm) that are formed by bacteria following a disruption in the normal division apparatus of bacterial cells. In essence, minicells are small, metabolically active replicas of normal bacterial cells with the exception that they contain no chromosomal DNA and as such, are non-dividing and non-viable. Although minicells do not contain chromosomal DNA, the ability of plasmids, RNA, native and/or recombinantly expressed proteins, and other metabolites have all been shown to segregate into minicells. Some methods of construction of minicell-producing bacterial strains are discussed in detail in U.S. patent application Ser. No. 10/154,951(US Publication No. US/2003/0194798 A1), which is hereby incorporated by reference in its entirety.

Disruptions in the coordination between chromosome replication and cell division lead to minicell formation from the polar region of most rod-shaped prokaryotes. Disruption of the coordination between chromosome replication and cell division can be facilitated through the overexpression of some of the genes involved in septum formation and binary fission. Alternatively, minicells can be produced in strains that harbor mutations in genes that modulate septum formation and binary fission. Impaired chromosome segregation mechanisms can also lead to minicell formation as has been shown in many different prokaryotes.

A description of methods of making, producing, and purifying bacterial minicells can be found, for example, in International Patent application No. WO2018/201160, WO2018/201161, and WO2019/060903, which are incorporated herein by reference.

Also, a description of strains for producing minicells can be found, for example, in International Patent application No. WO2018/201160, WO2018/201161, and WO2019/060903, which are incorporated herein by reference.

In some embodiments, the present disclosure teaches a composition comprising: a minicell and an active agent. In some embodiments, the minicell is derived from a bacterial cell. In some embodiments, the minicell is less than or equal to 1 μm in diameter.

The minicell is about 10 nm-about 1000 nm in size, about 50 nm-about 950 nm in size, about 100 nm-about 950 nm in size, about 150 nm-about 950 nm in size, or about 200 nm-about 900 nm in size. In other embodiments, the minicell is about 250 nm-about 900 nm in size. In further embodiments, the average minicell size produced from a controlled bioprocessing system of the present disclosure is about 400-about 750 nm, about 450-about 700 nm and about 500-about 650 nm in length.

### Agricultural Agents

The present disclosure provides coating platforms, compositions, formulations, methods for preparing a multilayer structure on an agricultural agents and products. In some embodiments, the agricultural agent is an agrochemical, a biologically active agent, or an agricultural product.

The present disclosure teaches that the agricultural agent is a pesticidal agent, an insecticidal agent, a herbicidal agent, a fungicidal agent, a virucidal agent, a nematicidal agent, a molluscicidal agent, an antimicrobial agent, an antibacterial agent, an antifungal agent, an antiviral agent, an antiparasitic agent, a fertilizing agent, a repellent agent, a plant growth regulating agent, or a plant-modifying agent.

In other embodiments, the agricultural agent is a nucleic acid, a polypeptide, a metabolite, a semiochemical, an essential oil, or a small molecule. In some embodiments, the nucleic acid is a DNA, an RNA, a PNA, or a hybrid DNA-RNA molecule. In some embodiments, the RNA is a messenger RNA (mRNA), a guide RNA (gRNA), or an inhibitory RNA. In some embodiments, the inhibitory RNA is RNAi, shRNA, or miRNA. In some embodiments, the inhibitory RNA inhibits gene expression in a plant. In some embodiments, the inhibitory RNA inhibits gene expression in a plant symbiont.

In some embodiments, the nucleic acid is an mRNA, a modified mRNA, or a DNA molecule that, in the plant, increases expression of an enzyme, a pore-forming protein, a signaling ligand, a cell penetrating peptide, a transcription factor, a receptor, an antibody, a nanobody, a gene editing protein, a riboprotein, a protein aptamer, or a chaperone.

In some embodiments, the nucleic acid is an antisense RNA, a siRNA, a shRNA, a miRNA, an aiRNA, a PNA, a morpholino, a LNA, a piRNA, a rib ozyme, a DNAzyme, an aptamer, a circRNA, a gRNA, or a DNA molecule that, in the plant, decreases expression of an enzyme, a transcription factor, a secretory protein, a structural factor, a riboprotein, a protein aptamer, a chaperone, a receptor, a signaling ligand, or a transporter.

In some embodiments, the polypeptide is an enzyme, pore-forming protein, signaling ligand, cell penetrating peptide, transcription factor, receptor, antibody, nanobody, gene editing protein, riboprotein, a protein aptamer, or chaperone.

A description of agricultural agents and active ingredients can be found, for example, in International Patent application Nos. WO2018/201160, WO2018/201161, WO2019/060903, and WO2021/133846, all of which are incorporated herein by reference.

### Agrochemical

In some embodiments, the agricultural agent is an agrochemical compound.

The term “agrochemical” as used herein means a chemical substance, whether naturally or synthetically obtained, which is applied to a plant, to a pest or to a locus thereof to result in expressing a desired biological activity. The term “biological activity” as used herein means elicitation of a stimulatory, inhibitory, regulatory, therapeutic, toxic or lethal response in a plant or in a pest such as a pathogen, parasite or feeding organism present in or on a plant or the elicitation of such a response in a locus of a plant, a pest or a structure. The term “plant” includes but shall not be limited to all food, fiber, feed and forage crops (pre and post harvest, seed and seed treatment), trees, turf and ornamentals. Examples of agrochemical substances include, but are not limited to, chemical pesticides (such as herbicides, algicides, fungicides, bactericides, viricides, insecticides, acaricides, miticides, rodenticides, nematicides and molluscicides), herbicide safeners, plant growth regulators (such as hormones and cell grown agents; including abscisic acid, auxin, brassinosteroid, cytokinin, ethylene, gibberellin, jasmonate, salicylic acid, strigolactone, plant peptide hormones, polyamine, nitric oxide, karrikin, triacontano etc.), fertilizers, soil conditioners, and nutrients, gametocides, defoliants, desiccants, mixtures thereof.

In some embodiments, the agrochemicals are synthetic or synthetically obtained. In other embodiments, the agrochemicals are naturally occurring or naturally obtained.

More examples of the above-described agrochemicals are described, for example, in U.S. Patent Application Publication Nos. US2012/0016022 and US2020/0113177, which are incorporated by reference herein in its entirety.

In some embodiments, an agricultural compound includes, but is not limited to a pesticide, an herbicide, an insecticide, a fungicide, a nematicide, a fertilizer, a nutrient, a plant growth regulator such as a hormone or a chemical growth agent, and any combination thereof.

### Biologically Active Agents

In some embodiments, the agricultural agent is a biologically active agent.

The term “biologically active agent” (synonymous with “bioactive agent”) indicates that an agent, a composition or compound itself has a biological effect, or that it modifies, causes, promotes, enhances, blocks, reduces, limits the production or activity of, or reacts with or binds to an endogenous molecule that has a biological effect. A “biological effect” may be but is not limited to one that impacts a biological process in/onto a plant; one that impacts a biological process in a pest, pathogen or parasite; one that generates or causes to be generated a detectable signal; and the like. Biologically active agents, compositions, complexes or compounds may be used in agricultural applications and compositions. Biologically active agents, compositions, complexes or compounds act to cause or stimulate a desired effect upon a plant, an insect, a worm, bacteria, fungi, or virus. Non-limiting examples of desired effects include, for example, (i) preventing, treating or curing a disease or condition in a plant suffering therefrom; (ii) limiting the growth of or killing a pest, a pathogen or a parasite that infects a plant; (iii) augmenting the phenotype or genotype of a plant; (iv) stimulating a positive response in a plant to germinate, grow vegetatively, bloom, fertilize, produce fruits and/or seeds, and harvest; and (v) controlling a pest to cause a disease or disorder.

In the context of agricultural applications of the present disclosure, the term “biologically active agent” indicates that the agent, composition, complex or compound has an activity that impacts vegetative and reproductive growth of a plant in a positive sense, impacts a plant suffering from a disease or disorder in a positive sense and/or impacts a pest, pathogen or parasite in a negative sense. Thus, a biologically active agent, composition, complex or compound may cause or promote a biological or biochemical activity within a plant that is detrimental to the growth and/or maintenance of a pest, pathogen or parasite; or of cells, tissues or organs of a plant that have abnormal growth or biochemical characteristics and/or a pest, a pathogen or a parasite that causes a disease or disorder within a host such as a plant.

In some embodiments, the biologically active agent is a natural product derived from a living organism. In some embodiments, the biologically active agent is a nucleic acid, a polypeptide, a protein, a metabolite, a semiochemical (such as pheromone), or an essential oil, which is a natural/naturally-occurring product or identical to a natural product. In some embodiments, the biologically active agents comprise biocontrols and biostimulants described below.

As one example of the biologically active agents, essential oils (EOs) such as peppermint oil (PO), thyme oil (TO), clove oil (CO), lemongrass oil (LO) and cinnamon oil (CnO) have been used for their antibacterial, antiviral, anti-inflammatory, antifungal, and antioxidant properties. Terpenoids such as menthol and thymol and phenylpropenes such as eugenol and cinnamaldehyde are components of EOs that mainly influence antibacterial activities. For example, thymol is able to disturb micromembranes by integration of its polar head-groups in lipid bilayers and increase of the intracellular ATP concentration. Eugenol was also found to affect the transport of ions through cellular membranes. Cinnamaldehyde inhibits enzymes associated in cytokine interactions and acts as an ATPase inhibitor.

In some embodiments, terpenes are chemical compounds that are widespread in nature, mainly in plants as constituents of essential oils (EOs). Their building block is the hydrocarbon isoprene (C5H8)n.

In some embodiments, examples of terpenes include, but are not limited to citral, pinene, nerol, b-ionone, geraniol, carvacrol, eugenol, carvone, terpeniol, anethole, camphor, menthol, limonene, nerolidol, framesol, phytol, carotene (vitamin A1), squalene, thymol, tocotrienol, perillyl alcohol, borneol, myrcene, simene, carene, terpenene, linalool and mixtures thereof. In some embodiments, the essential oil comprises geraniol, eugenol, genistein, carvacrol, thymol, pyrethrum or carvacrol.

In some embodiments, the essential oils can include oils from the classes of terpenes, terpenoids, phenylpropenes and combinations thereof. Essential oils as provided herein also contain essential oils derived from plants (i.e., “natural” essential oils) and additionally or alternatively their synthetic analogues.

It should be noted that terpenes are also known by the names of the extract or essential oil which contain them, e. g. peppermint oil (PO), thyme oil (TO), clove oil (CO), lemongrass oil (LO) and cinnamon oil (CnO).

In some embodiments, the biologically active agent is a nutrient including carbohydrates, fats, fiber, minerals, proteins, carbohydrates, fibers, vitamins, antioxidants, essential oils, and water. Examples of key nutrients for animal health can be classified as (i) proteins and amino acids (such as arginine, histidine, isoleucine, leucine, lysine, methionine, phenylalanine, threonine, tryptophan, valine, taurine, collagen and gelatin), (ii) fats (such as triglycerides, omega-3, omega-6, or omega-9 fatty acids, linoleic acid, tocopherols, arachidonic acid, docosahexaenoic acid (DHA), eicosapentaenoic acid (EPA)), (iii) carbohydrates (glucose, galactose, and fructose, lactose, disaccharides and oligosaccharides), (iv) fibers (cellulose and its derivatives, polysaccharides, and glycosaminoglycans), (v) vitamins (A, B-complex, D, C, E, K, thiamine and β-carotene), (vi) minerals (macrominerals such as sodium, potassium, calcium, phosphorus, magnesium), (vii) trace minerals of known importance such as iron, zinc, copper, iodine, fluorine, selenium, chromium, (viii) other minerals useful for animal nutrition such as cobalt, molybdenum, cadmium, arsenic, silicon, vanadium, nickel, lead, tin and (ix) antioxidants such as ascorbic acid, polyphenols, tannins, flavonols and triterpenes glucosides.

By way of non-limiting example, the biologically active agent or compound is a nucleic acid, a polypeptide, a metabolite, a semiochemical or a micronutrient. These biologically active agents can be broadly categorized as biocontrols and biostimulants.

(i) Biocontrols

The present disclosure teaches the biologically active agents as a biocontrol including, but are not limited to, a pesticide, an insecticide, a herbicide, a fungicide, a nematicide, an essential oil, an antimicrobial agent, an antifungal agent, and an antiviral agent.

In some embodiments, a pesticide, an insecticide, a herbicide, a fungicide, a nematicide, an antimicrobial agent, an antifungal agent, and an antiviral agent are natural products or naturally occurring agents produced by a living organism.

The present disclosure teaches the biologically active agents as a biocontrol including, but are not limited to, RNAi, protoxins, metabolites, antibodies, fermentation products, hormones, pheronomes, and semiochemicals. In some embodiments, biochemical control agents include, but are not limited to, semichemicals for example, plant-growth regulators, hormones, enzymes, pheromones, allomones and kairomones, which are either naturally occurring or identical to a natural product, that attract, retard, destroy or otherwise exert a pesticidal activity. In the some embodiments, biocontrols refer to biologically active compounds a polypeptide, a metabolite, a semiochemical, a hormone, a pheromone, and a nucleic acid such as RNA biomolecule including antisense nucleic acid, dsRNA, shRNA, siRNA, miRNA, ribozyme, and aptamer.

In some embodiments, semiochemicals includes pheromones, allomones, kairomones, and synomones. For example, pheromones, a class of microbial volatile organic compounds, can act as attractants and repellents to insects and other invertebrates. They can be used as biocontrol agents to control various pathogens as well as biofertilizers used for plant growth promotion. They are even used postharvest to prevent plant disease (Kanchiswamy et al., Trends Plant Sci. 40(4):206-211, 2015). Pheromones can be naturally produced or synthetically produced. Pheromones can be used for plant growth promotion. Some pheromones, derived from microorganisms, are able to promote the growth of some plants under various stressful conditions. For example, 2,3 butanediol, which is derived from the genus Bacillus) has been shown to induce systemic resistance and promote the growth of plants under stressful conditions like high salinity (Ryu et al., Plant Physiol. 134(3):1017-1026, 2004; Ryu et al., PNAS 100(8):4927-2932, 2003). Pheromones can be also used for pest management. Certain pheromones, usually derived from insects, are able to be used as biocontrol agents. They can be a part of a formulation that can attract and kill the target pest or they can be used for “mass-trapping of pest populations (Witzgall et al., J Chem Ecol. 36(1):80-100, 2010). For example, pheromones ((Z)-9-hexadecenal, (Z)-11-hexadecenal and (Z)-9-octadecenal, components of the S. incertulas pheromone) have been demonstrated to be able to control the population of yellow stem borer (Scirpophaga incertulas) on rice (Cork et al., Bulletin of Entomological Research, 86(5):515-524).

(ii) Biostimulants

The present disclosure teaches the biologically active agents as a biostimulant. Non-limiting examples of these biostimulants include hormones and biochemical growth agents. These actives include abscisic acid (involved in dormancy mechanisms under stress), auxins (positively influence plant growth), cytokinins (influence cell division and shoot formation), ACC Deaminase (lowers inhibitory growth effects of ethylene), gibberellins (positively influence plant growth by elongating stems and stimulating pollen tube growth), and many others (brassinosteroids, salicylic acid, jasmonates, plant peptide hormones, polyamines, nitric oxide, strigolactones, karrikins, and triacontanol), which are used to both positively and negatively regulate the growth of plants.

In some embodiments, the biologically active compounds are pheromones to improve and modify chemical reactions to help the plants grow and fight stresses as biostimulants.

In some embodiments, the biologically active agents are fertilizers, plant micronutrients and plant macro-nutrients, which include, but are not limited to, nitrogen, potassium, and phosphorous, and trace nutrients such as iron, copper, zinc, boron, manganese, calcium, molybdenum, and magnesium.

In some embodiments, biostimulants comprises microbial properties such as rhizobium (PGPRs) properties, fungal properties, cytokinins, phytohormones, peptides, and ACC-Deaminase. For example, nitrogen fixation can be achieved by delivering deliver ureases and/or nitrogenases via minicells to assist with nitrogen fixation.

In some embodiments, biostimulants comprises acids (such as humic substances, humin, fulvic acids, B vitamins, amino acids, fatty acids/lipids), extracts (such as carboxyls, botanicals, allelochemicals, betaines, polyamines, polyphenols, chitosan and other biopolymers), phosphites, phosphate solubilizers, nitrogenous compounds, inorganic salts, protein hydrolysates, and beneficial elements.

As one example, protein hydrolysates have potential to increase germination, productivity and quality of wide range of crops. Protein hydrolysates can also alleviate negative effects of salinity, drought, and heavy metals. Protein hydrolysates can stimulate carbon and nitrogen metabolism, and interfering with hormonal activity. Protein hydrolysates can enhance nutrient availability in plant growth substrates and increase nutrient uptake and use efficiency in plants. Protein hydrolysates can also stimulate plant microbiomes; substrates such as amino acids provided by protein hydrolysates could provide food source for plant-associated microbes.

Biostimulants foster plant development in a number of demonstrated ways throughout the crop lifecycle, from seed germination to plant maturity. They can be applied to plant, seed, soil or other growing media that may enhance the plant's ability to assimilate nutrients and properly develop. By fostering complementary soil microbes and improving metabolic efficiency, root development and nutrient delivery, biostimulants can increase yield in terms of weight, seed and fruit set, enhance quality, affecting sugar content, color and shelf life, improve the efficiency of water usage, and strengthen stress tolerance and recovery. These biostimulants can include pheromones or enzymes like ACC-Deaminase.

Biostimulants are compounds that produce non-nutritional plant growth responses and reduce stress by enhancing stress tolerance. Fertilizers, which produce a nutritional response can be considered as biostimulants. Many important benefits of biostimulants are based on their ability to influence hormonal activity. Hormones in plants (phytohormones) are chemical messengers regulating normal plant development as well as responses to the environment. Root and shoot growth, as well as other growth responses are regulated by phytohormones. Compounds in biostimulants can alter the hormonal status of a plant and exert large influences over its growth and health. Sea kelp, humic acids and B Vitamins are common components of biostimulants that are important sources of compounds that influence plant growth and hormonal activity. Antioxidants are another group of plant chemicals that are important in regulating the plants response to environmental and chemical stress (drought, heat, UV light and herbicides). When plants come under stress, “free radicals” or reactive oxygen molecules (e.g., hydrogen peroxide) damage the plants cells. Antioxidants suppress free radical toxicity. Plants with the high levels of antioxidants produce better root and shoot growth, maintain higher leaf-moisture content and lower disease incidence in both normal and stressful environments. Applying a biostimulant enhances antioxidant activity, which increases the plant's defensive system. Vitamin C, Vitamin E, and amino acids such as glycine are antioxidants contained in biostimulants.

Biostimulants may act to stimulate the growth of microorganisms that are present in soil or other plant growing medium. Biostimulants are capable of stimulating growth of microbes included in the microbial inoculant. Thus, it is desirable to obtain a biostimulant, that, when used with a microbial inoculant, is capable of enhancing the population of both native microbes and inoculant microbes.

In some embodiments, biologically active compounds can be used as biocontrols and biostimulants that have become the new age of crop protection and enhancement.

An example of a biocontrol is RNAi, or RNA interference, which is used to silence genes in target pests, killing them while leaving the non targeted pests unharmed. In invertebrates, long dsRNA can be efficiently used to silence gene expression without activation of dsRNA-activated protein kinase (PKR) or the interferon response that has been shown to occur in mammalian cell systems.

Another example is delivering protein toxins to combat pests. One example of protein toxin is orally active insecticidal peptide-1 (OAIP-1), which is to be highly toxic to insects with potency similar to that of the synthetic insecticide imidacloprid. This OAIP-1 toxin can be isolated from the Venom of an Australian Tarantula, which can be used as one of biologically active compounds taught in this disclosure.

Chitinases can be delivered to plants as a fungicide.

Plant antibodies are another form of biocontrols that can be used to specifically target pests. Immunoglobublin domains, light chain, heavy chain, and CDRs, Fv, Fab, and Fc regions can be encapsulated as active compounds and be delivered to a target. The present disclosure provides fungicidal antibodies such as those generated from glucosylceramide.

Plant-growth regulators, hormones, enzymes, pheromones, allomones and kairomones are also biocontrols. A pheromone can act as a biocontrol to prevent bugs and/or insects from mating.

Biostimulants foster plant development in a number of demonstrated ways throughout the crop lifecycle, from seed germination to plant maturity. They can be applied to plant, seed, soil or other growing media that may enhance the plant's ability to assimilate nutrients and properly develop. By fostering complementary soil microbes and improving metabolic efficiency, root development and nutrient delivery, biostimulants can increase yield in terms of weight, seed and fruit set, enhance quality, affecting sugar content, color and shelf life, improve the efficiency of water usage, and strengthen stress tolerance and recovery. These biostimulants can include pheromones or enzymes like ACC-Deaminase.

Biostimulants are compounds that produce non-nutritional plant growth responses and reduce stress by enhancing stress tolerance. Fertilizers, which produce a nutritional response can be considered as biostimulants. Many important benefits of biostimulants are based on their ability to influence hormonal activity. Hormones in plants (phytohormones) are chemical messengers regulating normal plant development as well as responses to the environment. Root and shoot growth, as well as other growth responses are regulated by phytohormones. Compounds in biostimulants can alter the hormonal status of a plant and exert large influences over its growth and health. Sea kelp, humic acids and B Vitamins are common components of biostimulants that are important sources of compounds that influence plant growth and hormonal activity.

Antioxidants are another group of plant chemicals that are important in regulating the plants response to environmental and chemical stress (drought, heat, UV light and herbicides). When plants come under stress, “free radicals” or reactive oxygen molecules (e.g., hydrogen peroxide) damage the plants cells. Antioxidants suppress free radical toxicity. Plants with the high levels of antioxidants produce better root and shoot growth, maintain higher leaf-moisture content and lower disease incidence in both normal and stressful environments. Applying a biostimulant enhances antioxidant activity, which increases the plant's defensive system. Vitamin C, Vitamin E, and amino acids such as glycine are antioxidants contained in biostimulants.

Biostimulants may act to stimulate the growth of microorganisms that are present in soil or other plant growing medium. Biostimulants are capable of stimulating growth of microbes included in the microbial inoculant. Thus, it is desirable to obtain a biostimulant, that, when used with a microbial inoculant, is capable of enhancing the population of both native microbes and inoculant microbes.

In some embodiments, the present disclosure provides an industrially suitable anucleated cell-based platform and/or an industrial formulation that can deliver biocontrols and biostimulants topically in a scalable, cost-effective manor by using the anucleated cell-based platform and/or an industrial formulation described herein. This anucleated cell-based platform and/or an industrial formulation can also be modified to invasively deliver biocontrols and biostimulants to plants including plants in aquaculture.

In one aspect, the anucleated cell-based platform and/or an industrial formulation uses bacterial cells lacking ribonucleases (ribonuclease III) and has T7, T3 or Sp6 RNA polymerase promoters to produce dsRNA used for RNA interference (RNAi) of a target. This bacterial cell is then modified to produce minicells with the dsRNA encapsulated within them. This helps simplify and cheapen purification and encapsulation. By encapsulating dsRNA, the dsRNA molecules are protected from environmental RNases. For examples, pests including insects orally consume the minicells for the delivery of the dsRNA. Once inside the insects, dsRNAs are a substrate for RNase III-like proteins referred to as Dicer or Dicer-like proteins. Dicer appears to preferentially initiate dsRNA cleavage at the ends of the dsRNA, making successive cleavages to generate 21- to 24-bp small-interfering (si) RNA duplexes to silence and/or suppress their target transcripts and inhibit translations of the transcripts. The resulting siRNA duplexes are loaded into a multiprotein complex called the RNA-induced silencing complex (RISC) where the passenger (sense) strand is removed and the guide (antisense) strand remains to target mRNA for silencing. The guide strand in the RISC enables base pairing of the complex to complementary mRNA transcripts and enzymatic cleavage of the target mRNA by a class of proteins referred to as Argonaute proteins, thereby preventing translation of the target mRNA. This is what causes the death of the targeted pest, while leaving untargeted pests unharmed. Also, the anucleated cell-based platform and/or an industrial formulation can be utilized to encapsulate dsRNA, siRNA shRNA, or miRNA. In other aspects, antisense nucleic acid, ribozyme, or aptamer can be encapsulated within the platform.

In some embodiments, the anucleated cell-based platform and the dsRNA are produced from different host cells and are incubated together after the independent productions have been completed. In some embodiments, the anucleated cell-based platforms can be utilized to internally express dsRNA from a recombinant plasmid capable of producing dsRNA inside of the anucleate minicell. Then, the internally produced dsRNA is delivered to its target within the anucleate minicell. In other embodiments, the anucleated cell-based platforms can be utilized to encapsulate externally and/or exogenously produced dsRNA that is first produced outside of the anucleate minicell. Then, the externally-produced dsRNA encapsulated into the minicell is delivered to its target within the anucleate minicell. In further embodiments, the anucleated cell-based platforms can be utilized to internally express dsRNA within the platform and encapsulate one or more sequences of exogenously-produced dsRNA into the platform for the purposes of targeting one or multiple different pests. This entails encapsulating dsRNA that is either homologous or heterologous to the internally expressed dsRNA sequence in the anucleate cell. Thus, the anucleated cell-based platform can carry both internally-expressed dsRNA and externally-expressed, but encapsulated dsRNA over to its intended target.

The present disclosure teaches that the anucleated cell-based platforms and/or an industrial formulation can deliver internally-produced dsRNA and externally/exogenously-produced dsRNA individually, or together to a target cell. The target cell is not a mammalian cell.

The present disclosure teaches that an industrially suitable anucleated cell-based platform and/or an industrial formulation for encapsulation and delivery of at least one biologically active compound, comprising: an intact anucleated cell derived from a ribonuclease deficient parental cell, comprising at least one biologically active compound within said cell, wherein said biologically active compound is a nucleic acid, wherein the nucleic acid targets a transcript encoding a polypeptide within a target cell, and wherein the target cell is not a mammalian cell. The anucleated cell-based platform and/or an industrial formulation further comprises at least one biologically acceptable carrier.

In some embodiments, for protein-mediated biocontrols, the present disclosure uses bacterial cells lacking proteases and has T7, T3, or Sp6 polymerase promoters to produce a significant amount of proteins. This bacterial cell is then modified to produce minicells with the proteins immobilized to their surface or encapsulated within them. A protein-expressing plasmid is integrated into the nucleoid DNA of the bacteria to safely and efficiently produce proteins. Insects then interact with or orally consume the minicells that express or retain the desired proteins. For antibody-mediated biocontrols, minicells can express or encapsulate antibodies to specifically target unwanted pests. Minicells can deliver antibodies or recombinant antibodies that serve as highly specific biopesticides against insects or fungal pathogens (Raymond et al., Fungal Biology Review 25(2):84-88, 2011).

In some embodiments, for biostimulants, the present disclosure teaches that minicells can deliver a wide range of plant-growth promoting biomolecules to the surface of the plant, its seeds, and its root system. Many of these biomolecules occur as a result of a dynamic, symbiotic relationship that some microorganisms have with plants and are produced naturally in response to certain environmental cues or stresses. The minicell can be engineered to deliver a high-payload capacity of these plant growth promoting biomolecules, either immobilized extracellularly on their surface or encapsulated intracellularly, without relying on microorganism or plants to naturally produce them. This enables a higher effective concentration of these biomolecules to be delivered to the plant microenvironment while also allowing for a more controlled, adaptive response to agricultural input needs. Many of these biomolecules are enzymes that bacteria produce, either intracellularly or extracellularly, that play an important role in promoting soil fertility and providing defense against plant pathogens (Jog et al, Journal of Appled Micorbiology 113:1154-1164, 2012; Sathya et al. 3 Biotech 7:102, 2017). Others, like 1-aminocyclopropane-1-carboxylate (ACC) Deaminase, can regulate plant growth on a hormonal level by lowering ethylene levels in the plant microenvironment (Souza et al., Genet. Mol. Biol. 38(4): 401-419, 2015).

In some embodiments, the biologically active compound are valuable enzymes that could be produced and delivered to the plant or its root system using the minicell, which include, but are not limited to cellulase, phytase, chitinase, protease, phosphatase, nucleases, lipases, glucanases, xylanases, amylases, peptidases, peroxidases, ligninases, pectinases, hemicellulases, and keratinases. Beyond being able to effectively deliver enzymes to promote the growth of plants, the minicell described herein can deliver other high-value biomolecules that play a role in promoting the growth of plants. These biomolecules include, but are not limited to plant hormones, such as the auxin IAA, peptides, primary metabolites, and secondary metabolites.

In some embodiments, the biologically active compounds are pheromones to improve and modify chemical reactions to help the plants grow and fight stresses as biostimulants.

In other embodiments, the delivery of biocontrols and biostimulants can be assisted through binding domains expressed on a surface of minicells. For example, minicells can express a binding domain such as a carbohydrate binding module (CBM) to be targeted to plants. These domains allow for better retention on plant surfaces, preventing runoff or drift. In some embodiments, minicells express a fusion protein comprising at least one surface expressing moiety and at least one target cell adhesion moiety, wherein said target cell adhesion moiety comprises a carbohydrate binding module. The target cell adhesion moiety comprises a carbohydrate binding module selected from the group consisting of: a cellulose binding domain, a xylan binding domain, a chitin binding domain, and a lignin binding domain.

In other embodiments, minicells can also express various proteins that encourage them to be uptaken by plants for invasive delivery through the leaf surface or roots. In some embodiments, minicells can express and display biologically active compound such as polypeptide and/or proteins on their surface. In other embodiments, minicells can express and display both surface expressed binding proteins and biologically active compound such as polypeptide and/or proteins on their surface.

The surface expressed binding proteins are as a carbohydrate binding module (CBM) described above. The biologically/enzymatically active polypeptide/proteins, which are surface-expressed, comprise cell stimulation moiety and/or cell degradation moiety. Non-limiting examples of such active proteins include, but are not limited to, ACC-deaminase, chitinase, cellulase, phytase, chitinase, protease, phosphatase, nucleases, lipases, glucanases, xylanases, amylases, peptidases, peroxidases, ligninases, pectinases, hemicellulases, and keratinases.

In some embodiments, these proteins are expressed exogenously and encapsulated into the minicells. In other embodiments, these proteins are internally expressed and immobilized on the surface of the minicells. The biologically active compounds such as such proteins are either encapsulated within the minicells after being expressed outside of the minicells or internally expressed within the minicells and displayed on the surface of the minicells. In further embodiments, the minicells express at least one biologically active compound on its surface and encapsulate another biologically active compound at the same time. So, the minicell can carry at least two biologically active compounds within the minicells and on the surface of the minicells. Non-limiting examples of such proteins include, but are not limited to ACC-deaminase, cellulase, phytase, chitinase, protease, phosphatase, nucleases, lipases, glucanases, xylanases, amylases, peptidases, peroxidases, ligninases, pectinases, hemicellulases, and keratinases.

In some embodiments, the protein is lipase used as a biocontrol compound. In other embodiments, the protein is lipase used as a biostimulant compound. In further embodiments, the protein is ACC deaminase used as a biostimulant compound. In some embodiments, the protein is lipase used as a biocontrol compound. In other embodiments, the protein is lipase used as a biostimulant compound. In further embodiments, the protein is ACC deaminase used as a biostimulant compound.

In some embodiments, minicells express a fusion protein comprising at least one surface expressing moiety and at least one target cell degradation moiety, wherein said target cell degradation moiety comprises an cutinase and cellulose.

The present disclosure teaches production and encapsulation of the RNA biomolecule including antisense nucleic acid, dsRNA, shRNA, siRNA, miRNA, ribozyme, or aptamer during the fermentation cycle by utilizing the microorganism's RNA synthesis and asymmetric division capabilities. This anucleated cell-based platform and/or an industrial formulation addresses three critical issues that have posed a great challenge to the delivery of ribonucleic acid (RNA) to a system: (1) the scalable synthesis and encapsulation of RNA (2) the synthesized/encapsulated oligonucleotide payload must survive the process; (3) the targeted delivery of this RNA biomolecule such that it reaches the tissue or cells of interest and invokes the desired phenotypic response. Current forms of RNA delivery are direct coupling of siRNA to N-acetylgalactosamine (GalNAc), formulating the RNA (often chemically modified) with cationic lipids and other excipients protects the oligonucleotide from the environment to compact its size, making chemical modifications to stabilize oligonucleotides for RNAi applications such as replacing the 2′-hydroxyl group on the ribose ring with 2′-methoxy and 2′-fluoro moieties. For dsRNA production, in vitro transcription is incredibly expensive compared to in vivo bacterial production of dsRNA. There are also Cell-Free and protein capsid processes for the production of dsRNA. The bacterial model is accompanied with the risk of environmental contamination due to proliferation of the modified species. This proliferation can have adverse and unforeseen consequences on the naturally existing species in the environment. Minicells result from naturally occurring mutations. The use of minicells for the purification and delivery of RNA allow for use the benefits of fermentation to scale the dsRNA production, without the risks associated with using genetically-modified bacteria. The use of minicells is also better for the delivery of protoxins and enzymes than using genetically-modified bacteria as biopesticides.

Payloads encapsulated in minicells produced from a controlled bioprocessing system of the present disclosure may be selected from a wide variety of agents, e.g., including biomolecules (including, e.g., enzyme, protein, carbohydrate, lipid, nucleic acid), agricultural agents including synthetic agrochemicals and biologically active agents taught herein. Agricultural agents may include, but not limited to, antibiotics, antivirals, antifungals, nucleic acids, plasmids, siRNAs, miRNA, antisense oligos, DNA binding compounds, hormones, vitamins, proteins, peptides, polypeptides. a pesticide, an insecticide, a herbicide, a fungicide, a nematicide, and an essential oil.

In some embodiments, said minicell is capable of encapsulating an agricultural agent. In some embodiments, said agricultural agent is an agrochemical compound or a biologically active compound. In some embodiments, said agrochemical compound is selected from the group consisting of: a pesticide, an herbicide, an insecticide, a fungicide, a nematicide, a fertilizer and a hormone or a chemical growth agent. In some embodiments, said biologically active compound is selected from a nucleic acid, a peptide, a protein, an essential oil, and combinations thereof. In some embodiments, the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof. In some embodiments, the essential oil comprises geraniol, eugenol, genistein, carvacrol, thymol, pyrethrum or carvacrol.

### Bioprocessing System

Mostly, the cultivation of bacteria or yeast well serves in shake flasks and cells in dishes or T-flasks Shake flasks, cell culture dishes, and T-flasks with many applications associated with cell culture. In the present disclosure, the cultivation for growing prokaryotic cells in the lab using shake flask systems is considered as an uncontrolled bioprocessing system.

The present disclosure teaches that bioreactors and fermenters are controlled bioprocessing systems that allow for larger quantities of cells/microbes/products, increased cultivation efficiency, improved product quality, and/or enhanced reproducibility of cell growth.

Broadly speaking, bioreactors and fermenters are culture systems to produce cells or organisms. They are used in various applications, including basic research and development, and the manufacturing of biopharmaceuticals, food and food additives, chemicals, and other products. A broad range of cell types and organisms can be cultivated in bioreactors and fermenters, including cells (like mammalian cell lines, insect cells, and stem cells), microorganisms (like bacteria, yeasts, and fungi), as well as plant cells and algae. Skilled ones in the art who cultivate bacteria, yeast, or fungi often use the term fermenter, while the term bioreactor often relates to the cultivation of mammalian cells.

In the present disclosure, bioreactor and fermenter are interchangeably used because they are basically referring to the same thing.

In some embodiments, the bioreactor refers to a cultivation vessel.

In some embodiments, the bioreactor is a 1-liter bioreactor, a 2-liter bioreactor, a 3-liter bioreactor, a 4-liter bioreactor, a 5-liter bioreactor, a 6-liter bioreactor, a 7-liter bioreactor, a 8-liter bioreactor, a 9-liter bioreactor, a 10-liter bioreactor, a 15-liter bioreactor, a 20-liter bioreactor, a 100-liter bioreactor, 1000-liter bioreactor, or 10,000-liter bioreactor, inclusive of all values and ranges therebetween.

In some embodiments, the bioreactor for commercial production is at least a 10,000-liter bioreactor, at least a 20,000-liter bioreactor, at least a 30,000-liter bioreactor, at least a 40,000-liter bioreactor, at least a 50,000-liter bioreactor, at least a 60,000-liter bioreactor, at least a 70,000-liter bioreactor, at least a 80,000-liter bioreactor, at least a 90,000-liter bioreactor, at least a 100,000-liter bioreactor, at least a 200,000-liter bioreactor, at least a 300,000-liter bioreactor, at least a 400,000-liter bioreactor, or at least a 500,000-liter bioreactor, inclusive of all values and ranges therebetween.

In other embodiments, the reactor is a bioreactor with working volumes of 1 ml to 250 ml therebetween. In further embodiments, the reactor is as small as 100 μl well on a multi-well microtiter plate, or even as small as microchip, or inclusive of all values and ranges therebetween.

In some embodiments, the size of the cultivation vessel depend upon the experimental parameters, such as number of cell types, number of media, number of different conditions to test, and the like. The skilled artisan can readily determine the appropriate cell cultivation vessel to employ.

In embodiments, cultivation vessel possibilities include, but are not limited to, cuvettes, culture plates such as 6-well plates, 24-well plates, 48-well plates and 96-well plates, culture dishes, microchips, 1-liter or larger bioreactors, cell culture flasks, roller bottles, culture tubes, culture vials, e.g., 3, 4 or 5 ml vials, flexible bags, and the like. The present disclosure teaches that any type of container can be used as a cultivation vessel.

In one embodiment, cell cultivation takes place in the bioreactor tank (a.k.a. a vessel) and the culture is mixed by stirring (instead of shaking). Stirred-tank bioreactors come in different sizes (for cultures of a few milliliters to thousands of liters).

In some embodiments, stirred-tank bioreactor can be used for minicell production of the present disclosure. The bioreactor is run by a controlled bioprocessing system, while the shaker or incubator is run by an uncontrolled bioprocessing system.

As used herein, “uncontrolled bioprocessing system” refers to a bioprocessing system in which culture or fermentation/culturing parameters (such as pH, temperature, dissolved oxygen, air flow rate, feed rate, growth rate etc.) are unable to control and maintain desired setpoints throughout the bioprocessing. A shaker or incubator for cell culture or biomaterial production is an example of the uncontrolled bioprocessing system. These fermentation/culturing parameters cannot be controlled in a shaker or incubator system.

As used herein, “controlled bioprocessing system” refers to a bioprocessing system in which culture or fermentation parameters (such as pH, temperature, dissolved oxygen, air flow rate, and feed rate) are controlled by continuously monitoring and consistently maintaining parameters at desired setpoints by sensors throughout the bioprocessing. A bioreactor or fermenter for cell culture is an example of the controlled bioprocessing system.

As used herein, “controlled continuous bioprocessing” refers to a bioprocessing that is continuously run by partial harvesting of the minicells from about 5 to about 95% of the batch and then being replenished with media to continue the fermentation. There is no lysis step involved with the controlled bioprocessing so the present disclosure teaches that the controlled bioprocessing system could run continuously for the minicell production. That is, the minicells are partially harvested from the batch and purified further steps, while the controlled bioprocess continues in the batch of the bioreactor.

The present disclosure teaches that the bioprocessing system taught herein is a controlled continuous bioprocessing system capable of continuously produce a population of achromosomal minicells. In some embodiments, said produced minicells are partially harvested and said bioprocessing system continuously run to produce another population of achromosomal minicells. In some embodiments, said minicells is partially harvested from about 1% to about 99%, about 2% to about 98%, about 3% to about 97%, about 4% to about 95%, about 5% to about 90% of total cells in the bioreactor.

Bioreactors allow for the creation of optimal environmental conditions for the growth of cells or microbes by culture mixing. In a bioreactor, the culture is stirred with an impeller. The present disclosure teaches that bacterial cell cultures are constantly mixed.

The temperature of the culture medium is controlled by continuously monitoring with a temperature sensor. The vessel is usually placed in a thermowell, wrapped with a heating blanket or has a water jacket. In embodiments, the temperature can be controlled between about 10° C. to about 70° C., about 15° C. to about 60° C., about 20° C. to about 50° C., about 25° C. to about 40° C., or about 30° C. to about 37° C. in the controlled bioprocessing system of the present disclosure. In further embodiments, the temperature can be controlled between about 15° C. to about 45° C., in the controlled bioprocessing system of the present disclosure.

Unlike a shaker or incubator, air or pure oxygen (e.g. coming from a compressed air cylinder) in bioreactors is usually introduced to the culture. Oxygen is important for culture growth, and the amount of oxygen dissolved in the medium (dissolved oxygen concentration, DO) is continuously measured with a DO sensor. In some embodiments, bioreactors are set to keep DO at setpoint. In embodiments, the dissolved oxygen (DO), that is the oxygen dissolved in given media, can be controlled between 0% to 100%, about 1% to about 99%, about 2% to about 98%, about 3% to about 97%, about 4% to about 96%, or about 5% to about 95% in the controlled bioprocessing system of the present disclosure.

In some embodiments, the minimum and maximum values of agitation, gas flow rate, and oxygen concentration can be optimized depending on the organism and process needs. In embodiments, the agitation can be controlled between about 50 to about 10,000 rpm or about 100 to about 9,000 rpm, or about 200 to about 8000 rpm in the controlled bioprocessing system of the present disclosure. In embodiments, the air flow rate can be controlled between about 0.1 to about 20 Standard liter per minute (SLPM), about 0.5 to about 15 SLPM, or about 1 to about 10 SLPM in the controlled bioprocessing system of the present disclosure. In embodiments, the oxygen that is supplied into a controlled bioprocess system can be controlled between 0% to 100%, about 1% to about 99%, about 2% to about 98%, about 3% to about 97%, about 4% to about 96%, or about 5% to about 95% in the controlled bioprocessing system of the present disclosure.

In some embodiments, oxygen is supplied to the system to maintain the defined value or range. In further embodiments, the oxygen is dissolved in media and the setting level of dissolved oxygen (DO) is maintained and is available for consumption by bacteria for growth or fermentation.

In bioreactors, the medium pH is continuously measured using a pH sensor and CO₂ is added as needed. For microbial cultures in bioreactors, basic and acid solutions are commonly used for pH adjustment. This is different from cultures in shake flasks, where the culture pH is usually not controlled. In embodiments, the pH can be controlled between 2-11 or 3-10 in the controlled bioprocessing system of the present disclosure.

Control of parameters at setpoint. In bioreactors, different components and the control software play together to control pH, temperature, and dissolved oxygen at the desired setpoint. The parameters are constantly measured using pH, temperature, and DO sensors. The sensors transmit the information to the bioprocess control software, which regulates the addition of CO₂ and liquid pH agents, the activity of tempering devices, agitation, and the gassing with air and/or O₂.

Bioreactors are used for a specific experiment or production run, which may last hours, days, or weeks, depending on the organism and application. In embodiments, the fermentation length can be controlled between 12 hours to 200 hours or 16 hours to 150 hours in the controlled bioprocessing system of the present disclosure.

A bioprocess run typically comprises the following steps:

(1) Preculture: The medium in the bioreactor is inoculated with a preculture. The preculture can be grown in a shaker or incubator, or in smaller bioreactors to grow precultures for the inoculation of larger bioreactors; (2) Bioreactor preparation: The bioreactor is prepared in parallel to inoculum preparation. Preparations include the sterilization of bioreactor, feed lines, and sensors; medium addition to the bioreactor; the connection of the bioreactor with the bioprocess control station; and the definition of process parameter setpoints in the bioprocess control software; (3) Inoculation: The medium is inoculated in the bioreactor; (4) Cultivation period (lag phase, exponential growth phase, stationary phase, stationary growth phase): Culture samples are taken to analyze the biomass and the concentration of metabolites. Eventually, the culture is fed by adding nutrient solutions; (5) Culture harvest: culture is harvested when the stationary growth phase reaches; (6) Downstream processing: The culture broth is further processed, including, but not limited to, that a batch of cells from the culture broth can be purified via disc stack centrifugation. The purified can be filtered for concentration. The purified batch of cells can be filtered (by cell size, length or diameter etc.) for concentration, then be stored as a liquid concentrate or dried down into a powder via freeze drying, vacuum drying, or heat drying; (7) Bioreactor cleaning: The bioreactor is sterilized to inactivate culture residues and cleaned. See Ulrike Rasche. International BioPharm: bioprocessing basics, June 2020 (eppendorf.com/product-media/doc/en/897339/Fermentors-Bioreactors_Publication_Bioprocess_Bioprocessing-basics.pdf)

The present disclosure teaches methods of bioprocessing, comprising the steps of: (a) introducing at least one bacterial cell strain into a bioreactor setting comprising minimal media; (b) culturing said bacterial cell strain from (a) to produce a population of achromosomal minicells in said bioreactor setting. In embodiments of the methods, said bacterial cell strain is a minicell-producing bacterial cell strain. In embodiments of the methods, said population of achromosomal minicells are produced from step (b); (c) harvesting a batch of cells comprising said bacterial cells and a population of newly-produced minicells from step (b); (d) purifying said batch of cells; (e) filtering or sorting out said population of achromosomal minicells from said batch of cells; and (f) concentrating said minicells. In embodiments, the purifying is performed by disc stack centrifugation. In embodiments, said concentrated minicells are stored as a liquid form or a powder form. In embodiments, said powder form is prepared by freeze-drying, vacuum drying, or heat drying of said concentrated minicells.

The bioprocessing system described above is not limited to cell cultivation. The present disclosure teaches that the bioprocessing system is utilized for minicell production in parallel to optimize conditions, determine parameters and/or conduct comparison studies.

In some embodiments, the bioprocessing system for minicell production is used for culturing large amounts of minicells.

The present disclosure teaches that bioreactors can allow for higher culture densities that can be achieved, while the growth of cells in flasks and dishes reaches a stationary phase without further increase further.

In some embodiments, growth-limiting factors such as nutrients, oxygen, and temperature control can be further supplied to microbial cell cultures in the bioreactors. In bioreactors, air or pure oxygen can be supplied by gassing, which is more efficient than shaking or agitation alone. If nutrients become limiting, growth stops. Cultures in bioreactors can quite easily and automatically fed by adding feed solutions using the system's integrated pumps.

Depending on the cell line or microbial strain, other parameters may be critical for culture growth and/or product formation, for example the medium pH, metabolite concentrations, redox potential, and mechanical forces. Bioreactors are valuable tools to optimize cultivation conditions.

The present disclosure teaches that bioreactors in a controlled bioprocessing system can increase reproducibility. In bioreactors, process parameters like pH, temperature, and dissolved oxygen can be constantly measured using sensors. The sensors transmit the information to the bioprocess control software, which regulates the action of actuators, like pumps, tempering devices, and gassing devices, to keep the parameters at setpoint. Monitoring, control, and recording of process values help increasing the reproducibility of culture growth, product formation, product characteristics, and more. In some embodiments, bioreactors in a controlled bioprocessing system can increase reproducibility of minicells produced from parental bacterial cells.

Bioprocess parameters including temperature, the medium pH, DO, metabolite concentrations, mechanical forces, and medium composition may influence culture growth and product yield (i.e. minicell yield). Process parameters influence cell viability, cell behavior, and differentiation. In a bioprocess, temperature, pH, and DO are routinely monitored and controlled. See Ulrike Rasche. International BioPharm: bioprocessing basics, June 2020.

The present disclosure teaches a bioprocessing system for minicell production, comprising: (a) at least one bioreactor; (b) a minimal medium for minicell production; and (c) at least one bacterial cell strain capable of producing a population of achromosomal minicells.

In embodiments, the minimal medium is commercially scalable by using cost-effective carbon and nitrogen sources with a minimal amount of other ingredients such as trace metals and vitamins, when comparing to a nutritionally-rich complex media such as LB or TB. The minimal media are used in the controlled bioprocessing system using the bioreactor or fermenter, and the complex media in the uncontrolled bioprocessing system using the shaker or incubator.

As used herein, “a minicell-producing cell” refers to a cell strain capable of producing a population of achromosomal minicells (i.e. minicells). In some embodiments, a minicell-producing bacterial cell refers to a bacterial cell capable of producing a population of achromosomal minicells (i.e. minicells).

In some embodiments of the bioprocessing system, said bioreactor is arranged for maintaining a continuous bioprocess configured to provide said population of achromosomal minicells.

In further embodiments of the bioprocessing system, a minicell production yield in said bioprocessing system is at least 1.1 fold, at least 1.2 fold, at least 1.3 fold, at least 1.4 fold, at least 1.5 fold, at least 2 fold, at least 3 fold, at least 4 fold, at least 5 fold, at least 6 fold, at least 7 fold, at least 8 fold, at least 9 fold, at least 10 fold, or at least 20 fold higher than a minicell production yield in an uncontrolled bioprocessing system.

In some embodiments, said uncontrolled bioprocessing system is an incubator system or a shaker flask system.

In some embodiments, a size range of said minicell is about 100 to about 900 nm, about 150 to about 800 nm, about 200 to about 700 nm, or about 250 to about 650 nm.

In some embodiments, a minicell ratio after said bioprocessing is at least 5%, at least 10%, at least 15%, at least 20%, at least 25%, at least 30%, at least 35%, at least 40%, at least 45%, at least 50%, at least 55%, at least 60% of total cells in a bioreactor.

### Cell Culture or Fermentation Parameters for Minicell Production

Growth rate, nutrient supply, biomass density and general principles uniquely controlled in a bioreactor coupled with a minicell producing strain conferring this high production yield can be disclosed herewith.

Minicell production is more analogous to biomolecule (such as protein) production than cell growth because minicell production is observed within defined parameters, whereas cell growth can occur in a constitutive minicell producing cell line without minicell production if not within appropriate parameters (temperature, dissolved oxygen, agitation, airflow rate etc.) as presented in Table 1.

TABLE 1
Fermentation Parameters for Minicell Production

| Feed rate for ingredients | 0-10 | mL/min/L |
|---|---|---|
| Temperature | 10-70° | C. |
| Ingredients | Carbon source (glycerol/glucose), trace | |
| | metals, vitamins, buffer, nitrogen source, | |
| | antifoam, additional growth | |
| | promoting/minicell production promoting | |
| | ingredients | |
| Dissolved oxygen (% DO) | 0-100% | |
| Agitation | 50-10,000 | rpm |
| Airflow rate | 0.1-20 | SLPM |
| Oxygen | 0-100% | |
| pH | 3-10 | |
| Inoculum | 0.1-20% | |
| Fermentation length | ~12-200 | h |

The present disclosure teaches a bioprocessing system for minicell production, comprising: (a) at least one bioreactor; (b) a minimal medium for minicell production; and (c) at least one bacterial cell strain capable of producing a population of achromosomal minicells.

In some embodiments of the bioprocessing system, said bioreactor is set with a fermentation parameter selected from the group of a feed rate, temperature, ingredients, dissolved oxygen, agitation speed, airflow rate, oxygen, pH, inoculum, and fermentation length.

In embodiments, said feed rate is 0 to about 10 mL/min/L. In embodiments, said temperature is from about 10° C. to about 70° C. In embodiments, said ingredients comprises a carbon source, a trace metal, a vitamin, a buffer, a nitrogen source, an antifoam, an additional growth promoting ingredient. In embodiments, said dissolved oxygen is 0 to 100%. In embodiments, said agitation speed is about 50 to about 10,000 rpm. In embodiments, said air flow rate is about 0.1 to about 20 standard liters per minute (SLPM). In embodiments, said oxygen is 0 to 100%. In embodiments, said pH is about 3 to about 10. In embodiments, said inoculum is about 0.1 to about 20%. In embodiments, said fermentation length is about 12 to about 200 hours. In some embodiments, said carbon source is a glycerol or a glucose.

Minicell production is analyzed by assessing auxotrophic and prototrophic minicell producing strains. Both types of minicell producing strains show an unexpected increase in minicell production once grown in a bioreactor based on the parameters described in Table 1. Prototrophic strains produce more minicells in a bioreactor setting.

The present disclosure teaches that an auxotrophic strain can be corrected, modified, mutated, or genetically engineered. The strains with their auxotrophy corrected react when grown in bioreactor conditions. In some embodiments, the strains with the auxotrophy corrected are able to be grown in fermentation parameters. The present disclosure provides that a significant increase in minicell production is observed in the given conditions not only from prototrophic strains, but also from strains with the auxotrophy corrected, modified, mutated, or genetically engineered.

Auxotrophs and prototrophs are alternative phenotypes. Auxotrophs are organisms that are unable to produce a particular organic compound required for their growth while prototrophs are organisms that can synthesize all organic compounds required for their growth from inorganic compounds.

In some embodiments, a minicell-producing bacterial cell strain can be an auxotrophic strain. In other embodiments, a minicell-producing bacterial cell strain can be an auxotrophic strain with its auxotropy corrected. The corrected or alleviated auxotrophic trait can obtained from genetic modification, mutation or gene/genome-editing technique to genes associated with auxotrophic traits.

In some embodiments, a minicell-producing bacterial cell strain can be a prototrophic strain.

In some embodiments, the minicell-producing bacteria is a Gram-negative bacteria. The Gram-negative bacteria includes, but is not limited to, Escherichia coli, Salmonella spp. including Salmonella typhimurium, Shigella spp. including Shigella flexneri, Pseudomonas aeruginosa, Agrobacterium, Campylobacter jejuni, Lactobacillus spp., Neisseria gonorrhoeae, and Legionella pneumophila,. In some embodiments, the minicell-producing gram-negative bacteria can produce minicells naturally caused by endogenous or exogenous mutation(s) associated with cell division and/or chromosomal partitioning. In some embodiments, the minicell-producing bacteria comprises endogenous or exogenous gene(s) that is involved in cell division and/or chromosomal partitioning, where the gene is genetically modified such as by homologous recombination, compared to a corresponding wild-type gene. In some embodiments, the minicell-producing gram-negative bacteria is deficient in protease and/or its activity naturally and/or by genetic engineering techniques. In some embodiments, the protease-deficient minicell-producing gram-negative bacteria comprises a recombinant expression vector comprising a gene or genes that is involved in a protein of interest disclosed in the present disclosure.

In some embodiments, a minicell-producing bacterial cell strain is derived from a bacterial genus selected from the group consisting of: Escherichia, Salmonella, Shigella, Pseudomonas, and Agrobacterium. In further embodiments, a minicell-producing bacterial cell strain is derived from from a bacterial species selected from the group consisting of: Escherichia coli, Salmonella typhimurium, Shigella flexneri, and Pseudomonas aeruginosa.

In some embodiments, minicell-producing bacterial cell strains (such as R1, P8-T7, and P826 strains) are a variety of strains derived from commercially available strains such as BL21 and P678-54. In some embodiments, minicell-producing bacterial cell strains (such as R1, P8-T7, and P826 strains) are a variety of strains produced by genetic modification(s), naturally-occurring or artificially induced mutation(s), a genetic engineering, or a gene/genome editing technique of commercially available strains such as BL21 and P678-54.

In some embodiments, the minicell-producing bacteria can be a Gram-positive bacteria. The Gram-positive bacteria includes, but is not limited to, Bacillus subtilis, Bacillus cereus, Corynebacterium Glutamicum, Lactobacillus acidophilus, Staphylococcus spp., or Streptococcus spp. In some embodiments, the minicell-producing gram-positive bacteria can produce minicells naturally caused by endogenous or exogenous mutation(s) associated with cell division and/or chromosomal partitioning. In some embodiments, the minicell-producing gram-positive bacteria comprises endogenous or exogenous gene(s) that is involved in cell division and/or chromosomal partitioning, where the gene is genetically modified such as by homologous recombination, compared to a corresponding wild-type gene. In some embodiments, the minicell-producing gram-positive bacteria is deficient in protease and/or its activity naturally and/or by genetic engineering techniques. In some embodiments, the protease-deficient minicell-producing gram-positive bacteria comprises a recombinant expression vector comprising a gene or genes that is involved in a protein of interest disclosed in the present disclosure.

In some embodiments, a minicell-producing bacterial cell strain is derived from a bacterial genus selected from the group consisting of: Bacillus, Corynebacterium, and Lactobacillus. In further embodiments, a minicell-producing bacterial cell strain is derived from from a bacterial species selected from the group consisting of: Bacillus subtilis, Corynebacterium glutamicum, and Lactobacillus acidophilus.

The present disclosure teaches a bioprocessing system for minicell production, comprising: (a) at least one bioreactor; (b) a minimal medium for minicell production; and (c) at least one bacterial cell strain capable of producing a population of achromosomal minicells.

### Methods for Bioprocessing in a Controlled Manner

The present disclosure teaches methods of bioprocessing, comprising the steps of: (a) introducing at least one bacterial cell strain into a bioreactor setting comprising minimal media; (b) culturing said bacterial cell strain from (a) to produce a population of achromosomal minicells in said bioreactor setting. In embodiments of the methods, said bacterial cell strain is a minicell-producing bacterial cell strain. In embodiments of the methods, said population of achromosomal minicells are produced from step (b). In embodiments of the methods, said bioreactor setting is configured with a fermentation parameter selected from the group of a feed rate, temperature, ingredients, dissolved oxygen, agitation speed, airflow rate, oxygen, pH, inoculum, and fermentation length. In embodiments of the methods, said produced minicell yield in said bioprocessing is at least 1.1 fold, at least 1.2 fold, at least 1.3 fold, at least 1.4 fold, at least 1.5 fold, at least 2 fold, at least 3 fold, at least 4 fold, at least 5 fold, at least 6 fold, at least 7 fold, at least 8 fold, at least 9 fold, at least 10 fold, or at least 20 fold higher than a minicell production yield in an uncontrolled bioprocessing.

In embodiments, the method comprises the steps of: (c) harvesting a batch of cells comprising said bacterial cells and a population of newly-produced minicells from step (b); (d) purifying said batch of cells; (e) filtering or sorting out said population of achromosomal minicells from said batch of cells; and (f) concentrating said minicells.

In embodiments, the purifying is performed by disc stack centrifugation. In embodiments, said concentrated minicells are stored as a liquid form or a powder form. In embodiments, said powder form is prepared by freeze-drying, vacuum drying, or heat drying of said concentrated minicells.

In embodiments, said bioprocessing is a controlled continuous bioprocessing capable of continuously producing a population of achromosomal minicells. In embodiments, said produced minicells are partially harvested and said bioprocessing system continuously run to produce another population of achromosomal minicells. In embodiments, said minicells is partially harvested from about 5% to about 90% of total cells in said bioreactor.

In embodiments of the methods, said feed rate is 0 to about 10 mL/min/L. In embodiments of the methods, said temperature is from about 10° C. to about 70° C. In embodiments of the methods, said ingredients comprises a carbon source, a trace metal, a vitamin, a buffer, a nitrogen source, an antifoam, an additional growth promoting ingredient. In embodiments of the methods, said dissolved oxygen is 0 to 100%. In embodiments of the methods, said agitation speed is about 50 to about 10,000 rpm. In embodiments of the methods, said air flow rate is about 0.1 to about 20 standard liters per minute (SLPM). In embodiments of the methods, said oxygen is 0 to 100%. In embodiments of the methods, said pH is about 3 to about 10. In embodiments of the methods, said inoculum is about 0.1 to about 20%. In embodiments of the methods, said fermentation length is about 12 to about 200 hours. In embodiments of the methods, said minicell is about 150 nm to about 950 nm in length. In embodiments of the methods, said carbon source is a glycerol or a glucose. In embodiments of the methods, said uncontrolled bioprocessing is an incubator setting or a shaker flask setting.

In embodiments of the methods, said minicell is capable of encapsulating an agricultural agent. In embodiments, said agricultural agent is an agrochemical compound or a biologically active compound. In embodiments, said agrochemical compound is selected from the group consisting of: a pesticide, an herbicide, an insecticide, a fungicide, a nematicide, a fertilizer and a hormone or a chemical growth agent. In embodiments, said biologically active compound is selected from a nucleic acid, a peptide, a protein, an essential oil, and combinations thereof. In embodiments, the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof. In embodiments, the essential oil comprises geraniol, eugenol, genistein, carvacrol, thymol, pyrethrum or carvacrol. In embodiments, a size range of said minicell is about 100 to about 900 nm, about 150 to about 800 nm, about 200 to about 700 nm, or about 250 to about 650 nm.

In embodiments, a minicell ratio after said bioprocessing is at least 5%, at least 10%, at least 15%, at least 20%, at least 25%, at least 30%, at least 35%, at least 40%, at least 45%, at least 50%, at least 55%, at least 60% of total cells in a bioreactor.

The present disclosure teaches a minicell produced by the method described herewith. In some embodiments, said minicell is produced in said bioprocessing setting at least 1.1 fold, at least 1.2 fold, at least 1.3 fold, at least 1.4 fold, or at least 1.5 fold higher than a minicell production yield in an uncontrolled bioprocessing system. In some embodiments, a fermentation parameter is listed in Table 1.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will occur to those skilled in the art.

### Example 1—Generation of Strain P6* from Strain P678-54

The E. coli strain P678-54 was auxotrophic according to experiments performed by inventors. See Adler et al., Genetic control of cell division in bacteria, 154 Science 417 (1966), and Adler et al. (Miniature Escherichia coli cells deficient in DNA, 57 Proc. Nat. Acad. Sci (Wash.) 321 (1967)) also supported by Adler 1966). Based on sequencing analysis, it was observed that the auxotrophic mutations in the genome of strain P678-54, relative to E. coli strain MG1655.

The genome of strain P678-54 was modified to correct the mutations in genes associated with auxotrophic traits in order to alleviate the auxotrophies. Natural directed evolution or gene/genome editing technology well known in the art can be used to correct target genes simultaneously.

The mutations were confirmed with DNA sequencing, and the auxotrophies were tested by plating on minimal media with and without leucine and/or threonine.

With the auxotrophy corrected, a new strain (P6*) was generated from P678-54. The P6* having minor genetic modification was used as a minicell producing strain like other minicell producing strains including R1.

The P6* strain can produce high level of minicells, and is able to grow in the defined bioreactor media recipe. As an auxotrophy is fixed in the P6* strain described above, its scalable growth and high minicell yield are achieved. The genetically-engineered P6* stain allows growth on defined media to enable cost effective scalable minicell production.

### Example 2—Minicell Production in an Uncontrolled Bioprocessing System

To understand minicell production yield in an uncontrolled bioprocessing system (i.e. a shaker flask system that was unable to control fermentation/culture parameters were not accurately controlled), a population of parental bacterial strain P6* capable of producing minicell as described in Example 1 was inoculated into undefined LB, TB, or similar complex media in a shaker flask system. The strain was incubated for 16 hours and minicell production yield was observed at 1 hour, 2 hours, 3 hours and 16 hours after inoculation. The temperature was set at 37° C. in the shaker flask system. In this example, a time course minicell production yield averaged for 4 independent runs and each time point averaged.

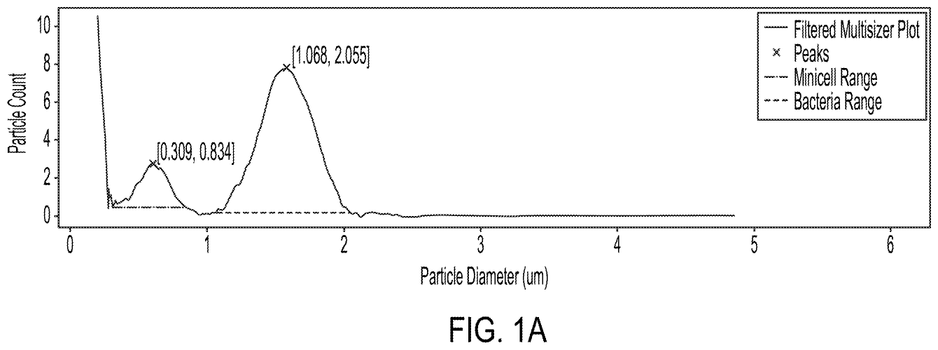

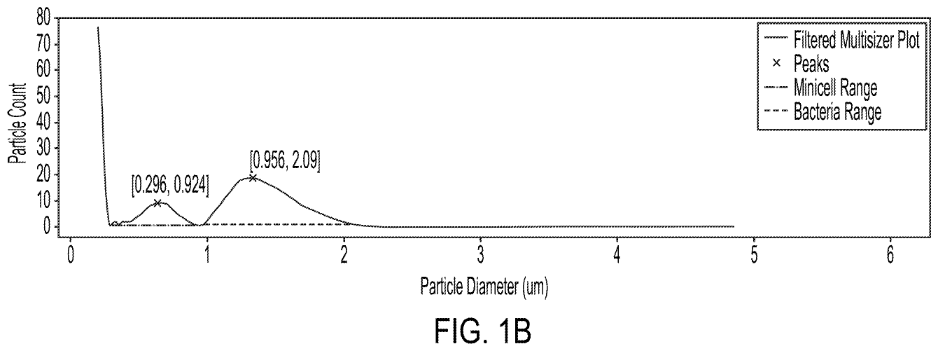

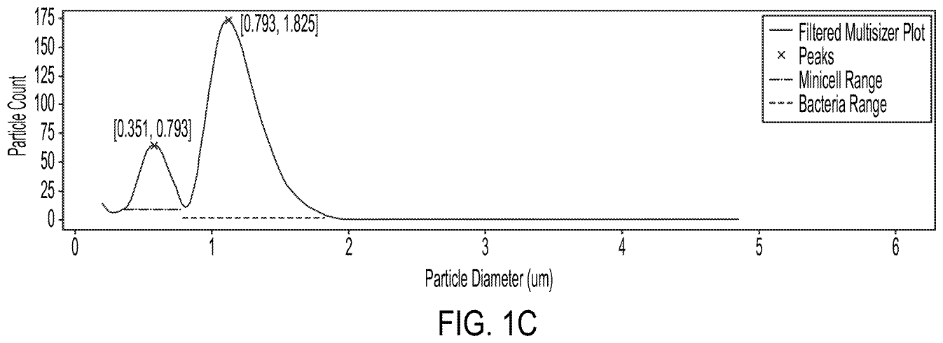

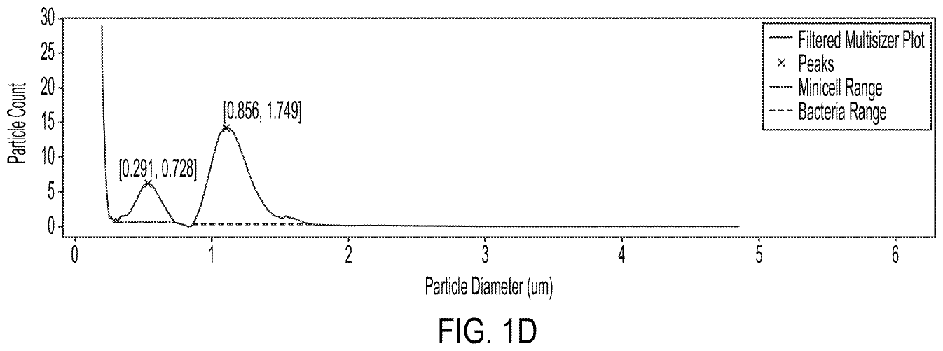

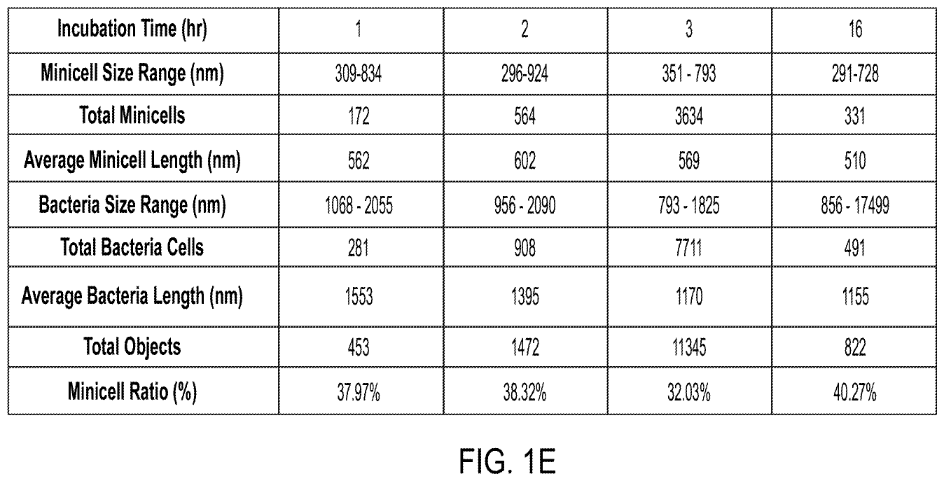

FIGS. 1A-1D shows time course of minicell production in un-controlled bioprocess (shaker flask system) grown in undefined complex LB, TB or similar complex media at 37° C. at hour 1 (FIG. 1A), hour 2 (FIG. 1B), hour 3 (FIG. 1C), and hour 16 (FIG. 1D). Newly-generated minicell population was detected at the first peak at ˜0.5 um capturing minicells and parental bacterial strain P6* was detected at the second peak at >1 um capturing the parent bacterial cells. Data from n=4 replicated runs. The minicell ratio (%) is calculated by a percentage of total minicells per total objects (both minicells and parental bacteria cells) from different incubation times (1 hour; 2 hours; 3 hours; and 16 hours). This example demonstrates that average minicell ratio is <40% when the uncontrolled bioprocessing system using a shaker was utilized as shown in FIG. 1E.

### Example 3—Minicell Production in a Controlled Bioprocessing System

To test enhanced minicell production yield in a controlled bioprocessing system (i.e. a bioreactor/fermenter system in which fermentation/culture parameters were accurately controlled and constantly maintained), a population of parental bacterial strain P6* capable of producing minicell was inoculated into defined minimal media in a bioreactor/fermenter system. The bioreactor/fermenter system was set with fermentation parameters for minicell production presented in Table 1. Minicell production yield was observed 36 hours after inoculation of the parental bacterial strain. The temperature was set at 37° C. in the bioreactor/fermenter system.

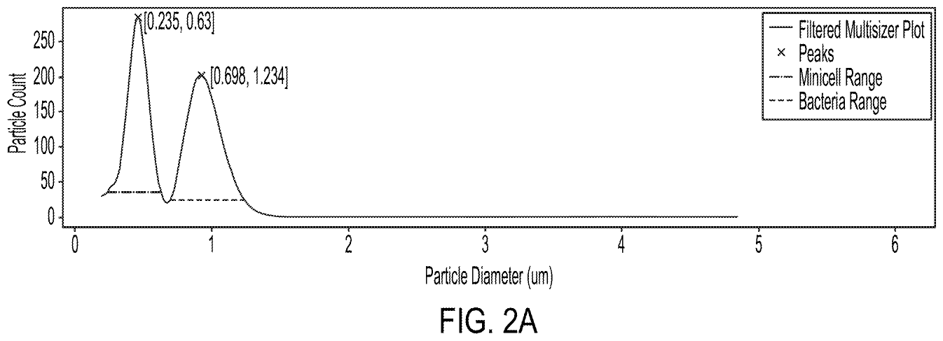

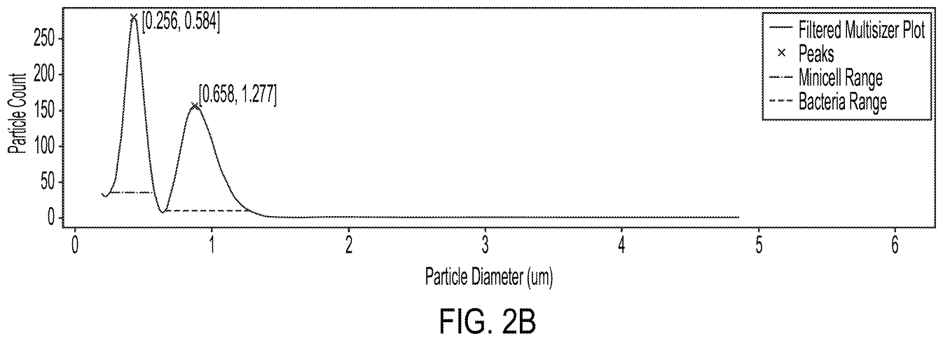

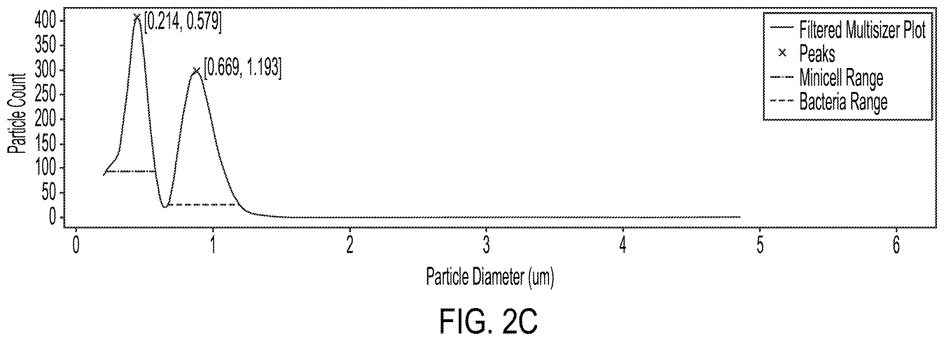

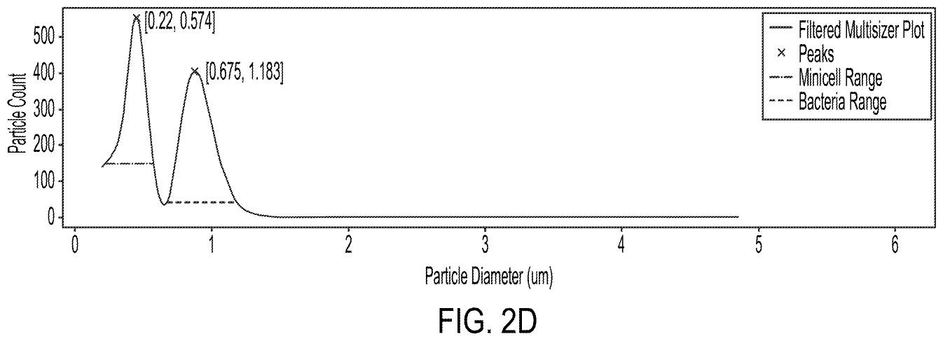

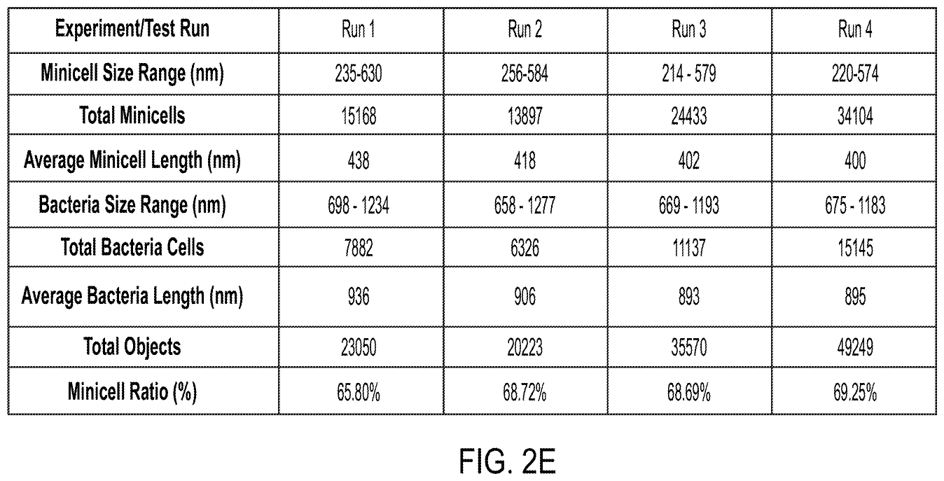

Minicell production was measured from four independent controlled bioprocess runs on different days; Run 1 (FIG. 2A), Run 2 (FIG. 2B), Run 3 (FIG. 2C), and Run 4 (FIG. 2D). FIGS. 2A-2D shows four individual runs for minicell production from P6* bacterial strain in controlled bioprocess (bioreactor/fermenter system) grown in the minimal media at 37° C. In FIGS. 2A-2D, the first peak is observed at −0.5 um capturing minicells and the second peak is observed at >1 um capturing the parent bacterial cells. Data from n=4 replicated runs as displayed in FIGS. 2A-2D. The minicell ratio (%) is calculated by a percentage of total minicells per total objects (both minicells and parental bacteria cells) at the end of fermentation time point (i.e. at 36 hours). This example demonstrates that average minicell ratio is >65% at different time points as shown in FIG. 2E.

These data points from 4 independent controlled bioprocess runs (FIG. 2E) showed consistency of enhanced minicell production when compared to the minicell production in the uncontrolled bioprocess runs (FIG. 1E). The minicell production yield in the controlled bioprocessing system (FIGS. 2A-2E) showed at least 1.5 fold higher than the minicell production yield in the uncontrolled bioprocessing system (FIGS. 1A-1E).

### Example 4—Effect of Varying Fermentation Parameters in a Controlled Bioprocessing System

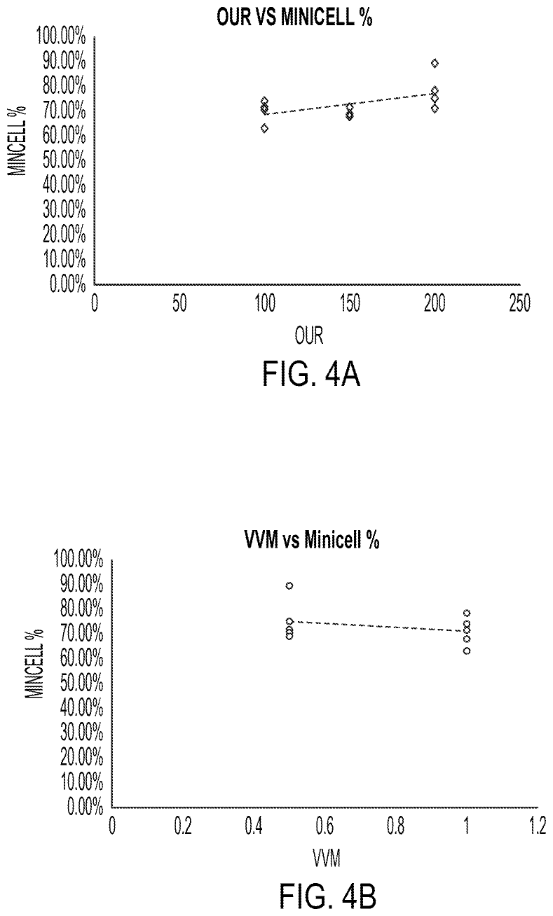

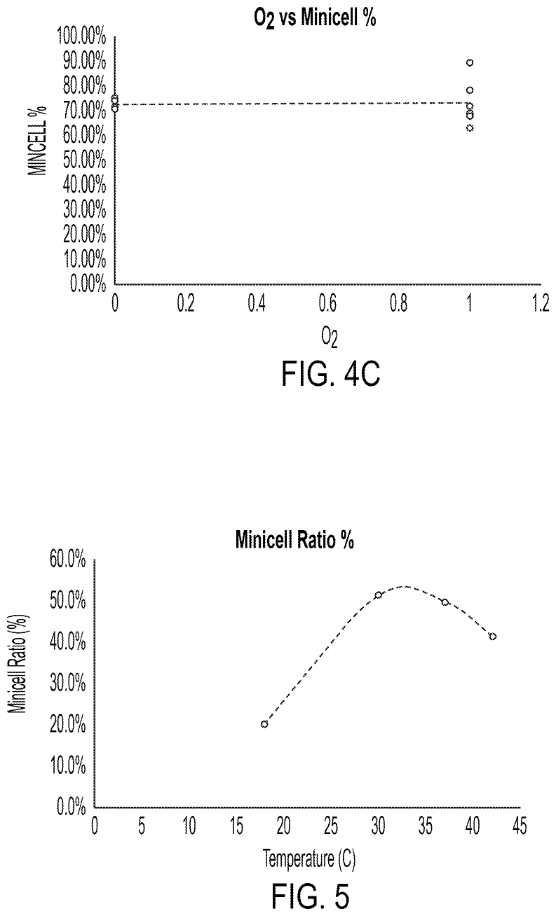

A varying range of fermentation parameters were tested to understand how various parameters affect minicell production. 12 individual bioreactor runs (BR1-12) were carried out in the controlled bioprocessing system with combinations of fermentation parameters such as Oxygen transfer (as measured by OUR) (FIG. 4A), airflow (VVM) (FIG. 4B) and supply of oxygen (02) (FIG. 4C). OUR is Oxygen Uptake Rate that is an indicator of how fast cells are growing. Max OD583 is a measure of max biomass P6* strain was used. Carbon source in the minimal medium for all 12 runs was glycerol.

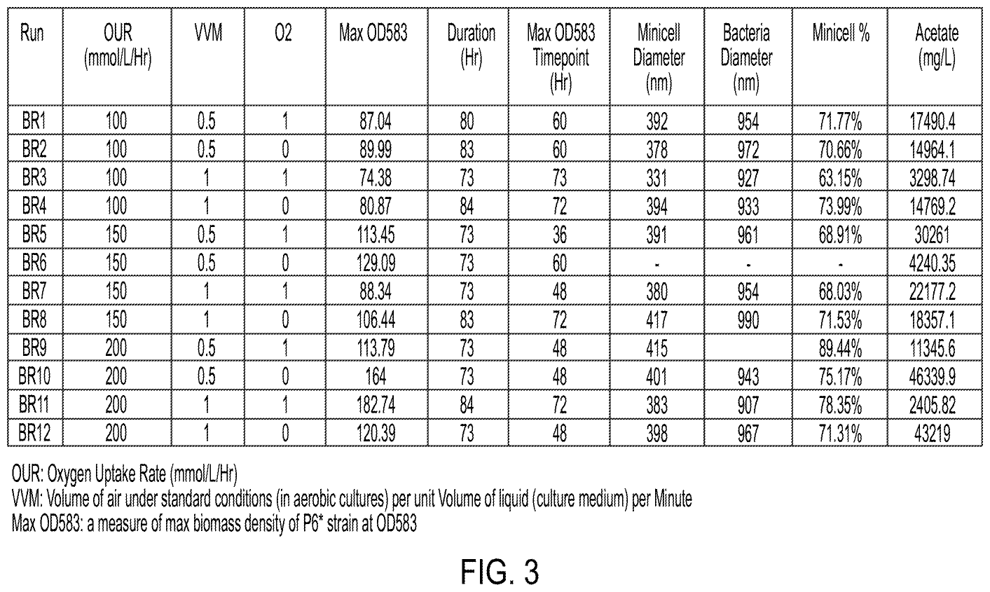

FIG. 3 presents enhanced minicell production (about 63% to about 89%) in a variety of parameter combinations of the controlled bioprocessing system, such as OUR, VVM, and 02 in the controlled bioprocess. Also, other outputs related to biomass (Max OD583 and Max OD583 timepoint) were observed in the 12 individual minicell production runs in the controlled bioprocess setting. Oxygen “0” means no additional Oxygen supplied other than normal air in the bioreactor and Oxygen “1” means supplementation of oxygen to enrich 02 level in the normal air. Data in FIG. 3 indicate that minicell production efficiencies are >63% in a variety of bioprocess control parameter combinations. The parameter variables are highly interlinked.

This experiment indicates that minicell production is enhanced in fermentation parameters defined in FIG. 3. However, the enhanced minicell production is not always correlated to enhanced biomass generation (Max OD583). Thus, the controlled bioprocess for enhanced minicell production described herewith is distinguished and different from a controlled bioprocess for exclusively biomass generation.

FIGS. 4A-4C show effect of various parameters on minicell production. Oxygen transfer/uptake (as measured by OUR) vs minicell production yield (FIG. 4A), airflow (VVM) vs minicell production yield (FIG. 4B) and supply of oxygen (O₂) vs minicell production yield (FIG. 4C). These data indicate that increased Oxygen transfer (as measured by OUR) is directly correlated with enhanced minicell production. However, VVM is not as strongly correlated with minicell production (%) as OUR.

### Example 5—Effect of Temperature in a Controlled Bioprocessing System

To test whether temperature affects minicell production in the controlled bioprocessing system, different temperature settings (from 15° C. to 45° C.) were set for strain P826 to produce minicells.

FIG. 5 shows that minicell ratio changes based on temperature and minicell production yield increases from about 18° C. until about 30° C. to 37° C.

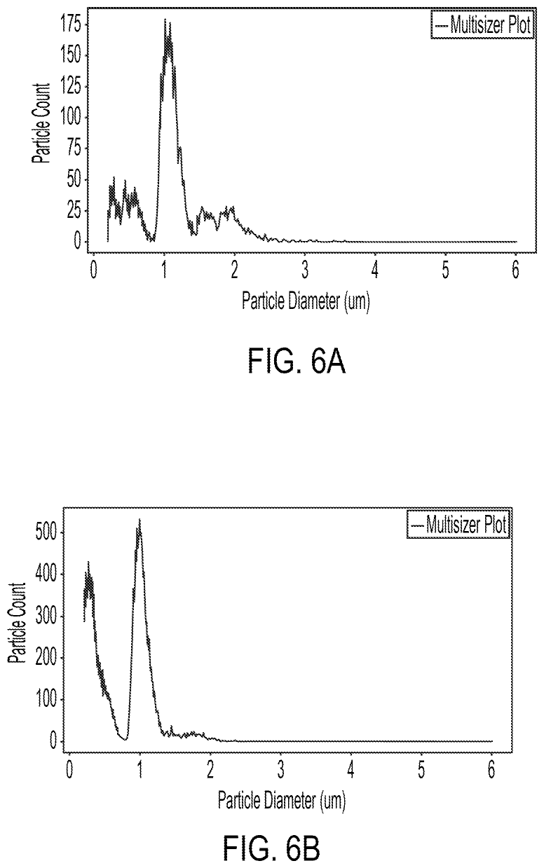

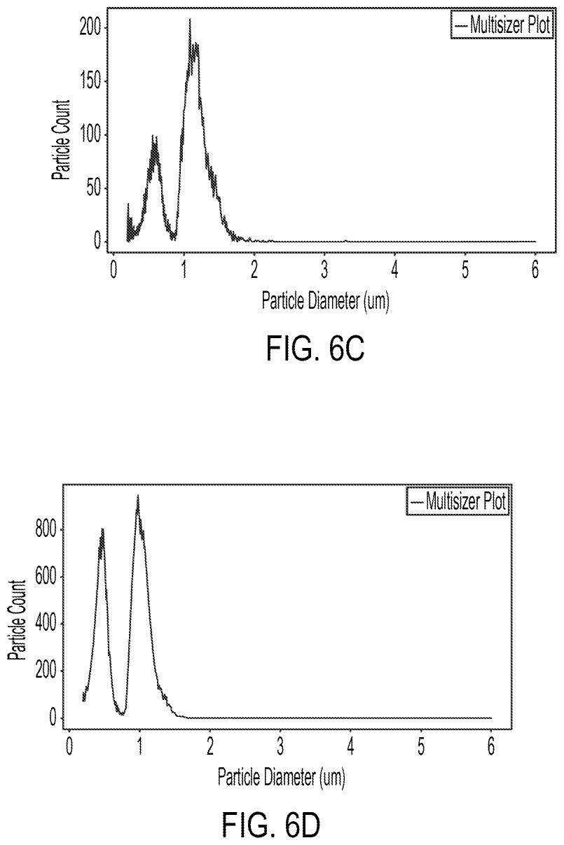

In order to compare minicell production yield from another minicell-producing strain (P826), P826 strain in complex media was inoculated and incubated at 25° C. for minicell production in an uncontrolled shaker flask system (FIG. 6A), while P826 strain in minimal media was inoculated and cultured at 25° C. for minicell production in a controlled bioreactor system at pH 6.7 and about 30% of DO (FIG. 6B). FIG. 6A indicates that minicells (1^{st} peak) were produced proportionally less than ⅓ of parental P826 cells (2^{nd} peak), while FIG. 6B shows about 1:1 ratio (or 0.8:1 ratio) of minicells (1^{st} peak) to parental P826 cells (2^{nd} peak). Also, quantity of produced minicells in FIG. 6B are at least 8 times more than those in FIG. 6A. FIG. 6A indicates that the minicell ratio is less than 25% from total number of cells (minicells and parental P826 cells), while FIG. 6B demonstrates enhanced minicell generation (at least 45% minicell ratio) in the controlled bioprocess system within the defined range of fermentation parameters.

FIGS. 6C-6D demonstrate the similar pattern of minicell generation, which had the same experiment setting except for the temperate set at 35° C. FIG. 6C indicates that minicells (1^{st} peak) were produced proportionally about half of parental P826 cells (2^{nd} peak), while FIG. 6D shows about 1:1 ratio (or 0.9:1 ratio) of minicells (1^{st} peak) to parental P826 cells (2^{nd} peak). Also, quantity of produced minicells in FIG. 6D are at least 8 times more than those in FIG. 6C. FIG. 6C indicates that the minicell ratio is less than 33% from total number of cells, while FIG. 6D demonstrates enhanced minicell generation (at least 45% minicell ratio) in the controlled bioprocess system within the defined range of fermentation parameters.

FIGS. 6B and 6D indicate enhancement of minicell production by utilizing control parameters listed in table 1 in the controlled bioprocessing system in comparison to FIGS. 6A and 6C.

### Example 6—Minicell Production from Bacterial Strains Expressing Biomolecules in a Controlled Bioprocessing System with Defined Fermentation Parameters

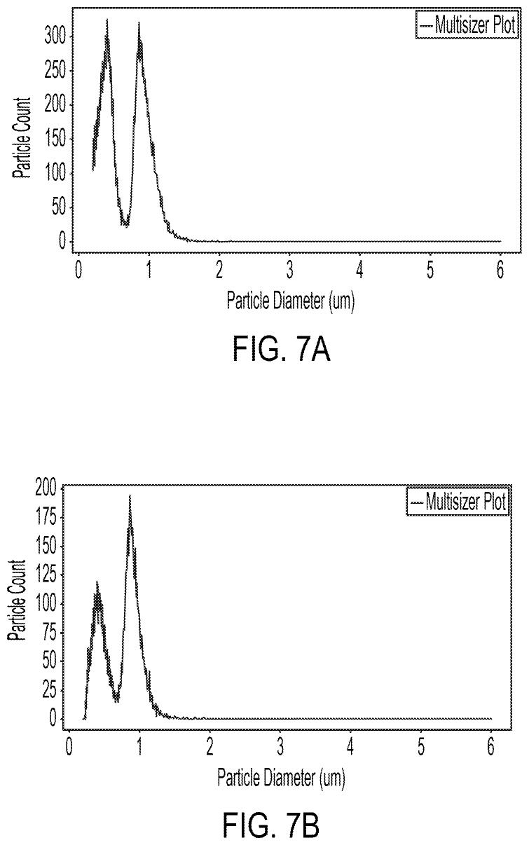

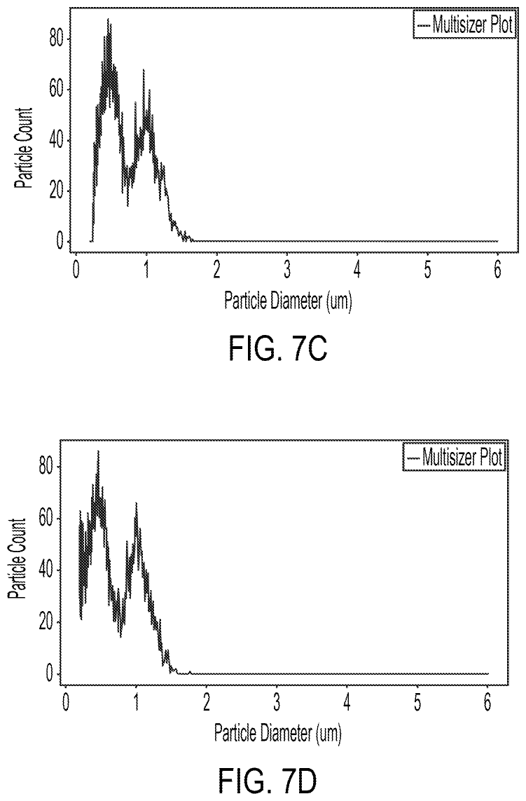

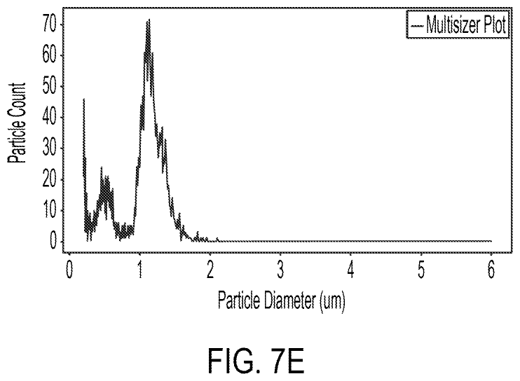

Minicell production from a biomolecule (RNA/Protein/metabolite) expression strains were tested in the controlled bioprocessing system within the defined range in Table 1. Two minicell-producing cell lines (P8-T7 and R1 bacterial strains), which also carry at least one construct expressing distinct double stranded RNA were used to test efficiency of minicell production in the controlled bioprocessing system. FIGS. 7A-7D show that enhanced minicell production from four individual strains carrying a different construct in the controlled bioprocessing system with the defined parameters listed in Table 1. Minicell productions are at least 50% of total cells (FIG. 7A), at least 40% of total cells (FIG. 7B), at least 65% of total cells (FIG. 7C) and at least 60% of total cells (FIG. 7D).

As a control, one of R1 strains carrying a construct expressing dsRNA to control insects was tested for efficiency of minicell production in the controlled bioprocessing system but outside of the defined parameters (i.e. pH was outside of the defined parameter range listed in Table 1 and not controlled). FIG. 7E shows that the minicell production from the strain is significantly lower (about 25% or less of total cells).

These data indicate that the defined parameters in Table 1 provide higher yield of minicell production even in the controlled bioprocessing system.

### NUMBERED EMBODIMENTS OF THE DISCLOSURE

Notwithstanding the appended claims, the disclosure sets forth the following numbered embodiments.
1. A bioprocessing system for minicell production, comprising:
   (a) at least one bioreactor;
   (b) a minimal medium for minicell production; and
   (c) at least one bacterial cell strain capable of producing a population of achromosomal minicells;
   wherein said bioreactor is arranged for maintaining a continuous bioprocess configured to provide said population of achromosomal minicells,
   wherein said bioreactor is set with a fermentation parameter selected from the group of a feed rate, temperature, ingredients, dissolved oxygen, agitation speed, airflow rate, oxygen, pH, inoculum, and fermentation length, and
   wherein a minicell production yield in said bioprocessing system is at least 1.1 fold higher than a minicell production yield in an uncontrolled bioprocessing system.
2. The bioprocessing system of embodiment 1, wherein said feed rate is 0 to about 10 mL/min/L.
3. The bioprocessing system of any one of embodiments 1-2, wherein said temperature is from about 10° C. to about 70° C.
4. The bioprocessing system of any one of embodiment s 1-3, wherein said ingredients comprises a carbon source, a trace metal, a vitamin, a buffer, a nitrogen source, an antifoam, an additional growth promoting ingredient.
5. The bioprocessing system of any one of embodiments 1-4, wherein said dissolved oxygen is 0 to 100%.
6. The bioprocessing system of any one of embodiments 1-5, wherein said agitation speed is about 50 to about 10,000 rpm.
7. The bioprocessing system of any one of embodiments 1-6, wherein said air flow rate is about 0.1 to about 20 standard liters per minute (SLPM).
8. The bioprocessing system of any one of embodiments 1-7, wherein said oxygen is 0 to 100%.
9. The bioprocessing system of any one of embodiments 1-8, wherein said pH is about 3 to about 10.
10. The bioprocessing system of any one of embodiments 1-9, wherein said inoculum is about 0.1 to about 20%.
11. The bioprocessing system of any one of embodiments 1-10, wherein said fermentation length is about 12 to about 200 hours.
12. The bioprocessing system of any one of embodiments 1-11, wherein said minicell is about 150 nm to about 950 nm in length.
13. The bioprocessing system of any one of embodiments 1-12, wherein said carbon source is a glycerol or a glucose.
14. The bioprocessing system of any one of embodiments 1-13, wherein said uncontrolled bioprocessing system is an incubator system or a shaker flask system.
15. The bioprocessing system of any one of embodiments 1-14, wherein said minicell is capable of encapsulating an agricultural agent.
16. The bioprocessing system of embodiment 15, wherein said agricultural agent is an agrochemical compound or a biologically active compound.
17. The bioprocessing system of embodiment 16, wherein said agrochemical compound is selected from the group consisting of: a pesticide, an herbicide, an insecticide, a fungicide, a nematicide, a fertilizer and a hormone or a chemical growth agent.
18. The bioprocessing system of embodiment 16, wherein said biologically active compound is selected from a nucleic acid, a peptide, a protein, an essential oil, and combinations thereof.
19. The bioprocessing system of embodiment 18, wherein the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof.
20. The bioprocessing system of embodiment 18, wherein the essential oil comprises geraniol, eugenol, genistein, carvacrol, thymol, pyrethrum or carvacrol.
21. The bioprocessing system of any one of embodiments 1-20, wherein a minicell ratio after said bioprocessing is at least 40% of total cells in a bioreactor.
22. The bioprocessing system of any one of embodiments 1-21, wherein said bioprocessing system is a controlled continuous bioprocessing system capable of continuously producing a population of achromosomal minicells.
23. The bioprocessing system of embodiment 22, wherein said produced minicells are partially harvested and said bioprocessing system continuously run to produce another population of achromosomal minicells.
24. The bioprocessing system of embodiment 23, wherein said minicells is partially harvested from about 5% to about 90% of total cells in said bioreactor.
25. A method of bioprocessing, comprising the steps of:
   (a) introducing at least one bacterial cell strain into a bioreactor setting comprising minimal media;
   (b) culturing said bacterial cell strain from (a) to produce a population of achromosomal minicells in said bioreactor setting,
   wherein said bacterial cell strain is a minicell-producing bacterial cell strain,
   wherein said population of achromosomal minicells are produced from step (b),
   wherein said bioreactor setting is configured with a fermentation parameter selected from the group of a feed rate, temperature, ingredients, dissolved oxygen, agitation speed, airflow rate, oxygen, pH, inoculum, and fermentation length, and
   wherein said produced minicell yield in said bioprocessing is at least 1.1 fold higher than a minicell production yield in an uncontrolled bioprocessing.
26. The method of embodiment 25, wherein said feed rate is 0 to about 10 mL/min/L.
27. The method of any one of embodiments 25-26, wherein said temperature is from about 10° C. to about 70° C.
28. The method of any one of embodiments 25-27, wherein said ingredients comprises a carbon source, a trace metal, a vitamin, a buffer, a nitrogen source, an antifoam, an additional growth promoting ingredient.
29. The method of any one of embodiments 25-28, wherein said dissolved oxygen is 0 to 100%.
30. The method of any one of embodiments 25-29, wherein said agitation speed is about 50 to about 10,000 rpm.
31. The method of any one of embodiments 25-30, wherein said air flow rate is about 0.1 to about 20 standard liters per minute (SLPM).
32. The method of any one of embodiments 25-31, wherein said oxygen is 0 to 100%.
33. The method of any one of embodiments 25-32, wherein said pH is about 3 to about 10.
34. The method of any one of embodiments 25-33, wherein said inoculum is about 0.1 to about 20%.
35. The method of any one of embodiments 25-34, wherein said fermentation length is about 12 to about 200 hours.
36. The method of any one of embodiments 25-35, wherein said minicell is about 150 nm to about 950 nm in length.
37. The method of any one of embodiments 25-36, wherein said carbon source is a glycerol or a glucose.
38. The method of any one of embodiments 25-37, wherein said uncontrolled bioprocessing is conducted in an incubator setting or a shaker flask setting.
39. The method of any one of embodiments 25-38, wherein said minicell is capable of encapsulating an agricultural agent.
40. The method of embodiment 39, wherein said agricultural agent is an agrochemical compound or a biologically active compound.
41. The method of embodiment 40, wherein said agrochemical compound is selected from the group consisting of: a pesticide, an herbicide, an insecticide, a fungicide, a nematicide, a fertilizer and a hormone or a chemical growth agent.
42. The method of embodiment 40, wherein said biologically active compound is selected from a nucleic acid, a peptide, a protein, an essential oil, and combinations thereof
43. The method of embodiment 42, wherein the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof
44. The method of embodiment 42, wherein the essential oil comprises geraniol, eugenol, genistein, carvacrol, thymol, pyrethrum or carvacrol.
45. The method of any one of embodiments 25-44, wherein a minicell ratio after said bioprocessing is at least 40% of total cells in a bioreactor.
46. The method of embodiment 25, further comprising the steps of:
   (c) harvesting a batch of cells comprising said bacterial cells and a population of newly-produced minicells from step (b);
   (d) purifying said batch of cells;
   (e) filtering or sorting out said population of achromosomal minicells from said batch of cells; and
   (f) concentrating said minicells.
47. The method of embodiment 46, wherein the purifying is performed by disc stack centrifugation.
48. The method of embodiment 46, wherein said concentrated minicells are stored as a liquid form or a powder form.
49. The method of embodiment 48, wherein said powder form is prepared by freeze-drying, vacuum drying, or heat drying of said concentrated minicells.
50. The method of embodiment 25 or 46, wherein said bioprocessing is a controlled continuous bioprocessing capable of continuously producing a population of achromosomal minicells.
51. The method of embodiment 25 or 46, wherein said produced minicells are partially harvested and said bioprocessing system continuously run to produce another population of achromosomal minicells.
52. The bioprocessing system of embodiment 51, wherein said minicells is partially harvested from about 5% to about 90% of total cells in said bioreactor.
53. A minicell produced by the method of embodiment 25 or 46.
54. The minicell of embodiment 53, wherein said minicell is produced in said bioprocessing setting at least 1.1 fold higher than a minicell production in an uncontrolled bioprocessing setting.
55. A fermentation parameter of embodiment 1, 25 or 46, wherein said fermentation parameter is listed in Table 1.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.
U.S. Pat. No. 3,467,604
U.S. Patent Application No. 2012/0016022
U.S. Patent Application No. 2012/0016022
U.S. Patent Application No. 2016/0174571
International Patent application No. WO 09/013361
International Patent application No. WO2018/201160
International Patent application No. WO2018/201161
International Patent application No. WO2019/060903
International Patent application No. WO2021/133846

1. A bioprocessing system for minicell production, comprising:
(a) at least one bioreactor;
(b) a minimal medium for minicell production; and
(c) at least one bacterial cell strain capable of producing a population of achromosomal minicells;
wherein said bioreactor is arranged for maintaining a continuous bioprocess configured to provide said population of achromosomal minicells,
wherein said bioreactor is set with a fermentation parameter selected from the group of a feed rate, temperature, ingredients, dissolved oxygen, agitation speed, airflow rate, oxygen, pH, inoculum, and fermentation length, and
wherein a minicell production yield in said bioprocessing system is at least 1.1 fold higher than a minicell production yield in an uncontrolled bioprocessing system.

2. The bioprocessing system of claim 1, wherein said feed rate is 0 to about 10 mL/min/L.

3. The bioprocessing system of claim 1, wherein said temperature is from about 10° C. to about 70° C.

4. The bioprocessing system of claim 1, wherein said ingredients comprises a carbon source, a trace metal, a vitamin, a buffer, a nitrogen source, an antifoam, or an additional growth promoting ingredient.

5. The bioprocessing system of claim 1, wherein said dissolved oxygen is 0 to 100%.

6. The bioprocessing system of claim 1, wherein said agitation speed is about 50 to about 10,000 rpm.

7. The bioprocessing system of claim 1, wherein said air flow rate is about 0.1 to about 20 standard liters per minute (SLPM).

8. The bioprocessing system of claim 1, wherein said oxygen is 0 to 100%.

9. The bioprocessing system of claim 1, wherein said pH is about 3 to about 10.

10. The bioprocessing system of claim 1, wherein said inoculum is about 0.1 to about 20%.

11. The bioprocessing system of claim 1, wherein said fermentation length is about 12 to about 200 hours.

12. The bioprocessing system of claim 1, wherein said minicell is about 150 nm to about 950 nm in length.

13. The bioprocessing system of claim 1, wherein said carbon source is a glycerol or a glucose.

14. The bioprocessing system of claim 1, wherein said uncontrolled bioprocessing system is an incubator system or a shaker flask system.

15. The bioprocessing system of claim 1, wherein said minicell is capable of encapsulating an agricultural agent.

16. The bioprocessing system of claim 15, wherein said agricultural agent is an agrochemical compound or a biologically active compound.

17. The bioprocessing system of claim 16, wherein said agrochemical compound is selected from the group consisting of a pesticide, an herbicide, an insecticide, a fungicide, a nematicide, a fertilizer, a hormone, a chemical growth agent, and combinations thereof.

18. The bioprocessing system of claim 16, wherein said biologically active compound is selected from a nucleic acid, a peptide, a protein, an essential oil, and combinations thereof.

19. The bioprocessing system of claim 18, wherein the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof.

20. The bioprocessing system of claim 18, wherein the essential oil comprises geraniol, eugenol, genistein, carvacrol, thymol, pyrethrum or carvacrol.

21. The bioprocessing system of claim 1, wherein a minicell ratio after said bioprocessing is at least 40% of total cells in a bioreactor.

22. The bioprocessing system of claim 1, wherein said bioprocessing system is a controlled continuous bioprocessing system capable of continuously producing a population of achromosomal minicells.

23. The bioprocessing system of claim 22, wherein said produced minicells are partially harvested and said bioprocessing system continuously run to produce another population of achromosomal minicells.

24. The bioprocessing system of claim 23, wherein said minicells is partially harvested from about 5% to about 90% of total cells in said bioreactor.

25. A method of bioprocessing, comprising the steps of:
(a) introducing at least one bacterial cell strain into a bioreactor setting comprising minimal media;
(b) culturing said bacterial cell strain from (a) to produce a population of achromosomal minicells in said bioreactor setting,
wherein said bacterial cell strain is a minicell-producing bacterial cell strain,
wherein said population of achromosomal minicells are produced from step (b),
wherein said bioreactor setting is configured with a fermentation parameter selected from the group of a feed rate, temperature, ingredients, dissolved oxygen, agitation speed, airflow rate, oxygen, pH, inoculum, and fermentation length, and
wherein said produced minicell yield in said bioprocessing is at least 1.1 fold higher than a minicell production yield in an uncontrolled bioprocessing.

26.-45. (canceled)

46. The method of claim 25, further comprising the steps of:
(c) harvesting a batch of cells comprising said bacterial cells and a population of newly-produced minicells from step (b);
(d) purifying said batch of cells;
(e) filtering or sorting out said population of achromosomal minicells from said batch of cells; and
(f) concentrating said minicells.

47.-49. (canceled)

50. The method of claim 25, wherein said bioprocessing is a controlled continuous bioprocessing capable of continuously producing a population of achromosomal minicells.

51. The method of claim 25, wherein said produced minicells are partially harvested and said bioprocessing system continuously run to produce another population of achromosomal minicells.

52. The bioprocessing system of claim 51, wherein said minicells is partially harvested from about 5% to about 90% of total cells in said bioreactor.

53. A minicell produced by the method of claim 25.

54.-55. (canceled)
